SEQUENCE LISTING (42 sequences)

SEQ ID NO: 1 - protein, 522 aa, Artificial Sequence
  Synthesized
MALPVTALLLPLALLLHAARPEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSTTTPAPRPPTPAPTIASQPLSLRPEACRPAAGGAVHTRGLDFACDIYIWAPLAGTCGVLLLSLVITCVRCRHRRRQAERMSQIKRLLSEKKTCQCPHRFQKTCSPIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 525 aa, Artificial Sequence
  Synthesized
MALPVTALLLPLALLLHAARPEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRCVRCRHRRRQAERMSQIKRLLSEKKTCQCPHRFQKTCSPI

SEQ ID NO: 3 - protein, 500 aa, Artificial Sequence
  Synthesized
MALPVTALLLPLALLLHAARPEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSSGQVLLESNIKVLPTWSTPVQPMALIVLGGVAGLLLFIGLGIFFCVRCRHRRRQAERMSQIKRLLSEKKTCQCPHRFQKTCSPIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 500 aa, Artificial Sequence
  Synthesized
MALPVTALLLPLALLLHAARPEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSSGQVLLESNIKVLPTWSTPVQPMALIVLGGVAGLLLFIGLGIFFKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRCVRCRHRRRQAERMSQIKRLLSEKKTCQCPHRFQKTCSPI

SEQ ID NO: 5 - protein, 853 aa, Artificial Sequence
  Synthesized
MALPVTALLLPLALLLHAARPEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 6 - protein, 21 aa, Artificial Sequence
  Synthesized
MALPVTALLLPLALLLHAARP

SEQ ID NO: 7 - protein, 45 aa, Homo sapiens
TTTPAPRPPTPAPTIASQPLSLRPEACRPAAGGAVHTRGLDFACD

SEQ ID NO: 8 - protein, 21 aa, Homo sapiens
IYIWAPLAGTCGVLLLSLVIT

SEQ ID NO: 9 - protein, 32 aa, Homo sapiens
LYCNHRNRRRVCKCPRPVVKSGDKPSLSARYV

SEQ ID NO: 10 - protein, 19 aa, Homo sapiens
HLCCRRRRARLRFMKQFYK

SEQ ID NO: 11 - protein, 9 aa, Homo sapiens
RVCKCPRPV

SEQ ID NO: 12 - protein, 22 aa, Homo sapiens
SGQVLLESNIKVLPTWSTPVQP

SEQ ID NO: 13 - protein, 22 aa, Homo sapiens
MALIVLGGVAGLLLFIGLGIFF

SEQ ID NO: 14 - protein, 40 aa, Homo sapiens
CVRCRHRRRQAERMSQIKRLLSEKKTCQCPHRFQKTCSPI

SEQ ID NO: 15 - protein, 17 aa, Homo sapiens
MSQIKRLLSEKKTCQCP

SEQ ID NO: 16 - protein, 481 aa, Artificial Sequence
  Synthesized
MALPVTALLLPLALLLHAARPQIVLTQSPAIMSASPGEKVTITCSVSSSVDYIHWFQQKPGTSPKFWIYSTSILASGVPARFSGSGSGTSYSLTISRMEAEDAATYYCQQRSSYPPTFGSGTKLEIKGGGGSGGGGSGGGGSQVQLQQSGGEVMKPGASVKLSCKATGYTFTGYWIEWIKQRPGHGLEWIGEILPGSGGTNYNEKFKGKATFTAHTSSNTAYMQLSSLTTEDSAIYYCARDSNSFTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 194 aa, Artificial Sequence
  Synthesized
CVRCRHRRRQAERMSQIKRLLSEKKTCQCPHRFQKTCSPIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 238 aa, Artificial Sequence
  Synthesized
SGQVLLESNIKVLPTWSTPVQPMALIVLGGVAGLLLFIGLGIFFCVRCRHRRRQAERMSQIKRLLSEKKTCQCPHRFQKTCSPIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 241 aa, Artificial Sequence
  Synthesized
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 20 - protein, 171 aa, Artificial Sequence
  Synthesized
MSQIKRLLSEKKTCQCPKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 186 aa, Homo sapiens
LYCNHRNRRRVCKCPRPVVKSGDKPSLSARYVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 173 aa, Artificial Sequence
  Synthesized
HLCCRRRRARLRFMKQFYKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 163 aa, Artificial Sequence
  Synthesized
RVCKCPRPVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 66 aa, Homo sapiens
TTTPAPRPPTPAPTIASQPLSLRPEACRPAAGGAVHTRGLDFACDIYIWAPLAGTCGVLLLSLVIT

SEQ ID NO: 25 - protein, 496 aa, Artificial Sequence
  Synthesized
MALPVTALLLPLALLLHAARPQIVLTQSPAIMSASPGEKVTITCSVSSSVDYIHWFQQKPGTSPKFWIYSTSILASGVPARFSGSGSGTSYSLTISRMEAEDAATYYCQQRSSYPPTFGSGTKLEIKGGGGSGGGGSGGGGSQVQLQQSGGEVMKPGASVKLSCKATGYTFTGYWIEWIKQRPGHGLEWIGEILPGSGGTNYNEKFKGKATFTAHTSSNTAYMQLSSLTTEDSAIYYCARDSNSFTYWGQGTLVTVSSSGQVLLESNIKVLPTWSTPVQPMALIVLGGVAGLLLFIGLGIFFCVRCRHRRRQAERMSQIKRLLSEKKTCQCPHRFQKTCSPIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 496 aa, Artificial Sequence
  Synthesized
MALPVTALLLPLALLLHAARPQIVLTQSPAIMSASPGEKVTITCSVSSSVDYIHWFQQKPGTSPKFWIYSTSILASGVPARFSGSGSGTSYSLTISRMEAEDAATYYCQQRSSYPPTFGSGTKLEIKGGGGSGGGGSGGGGSQVQLQQSGGEVMKPGASVKLSCKATGYTFTGYWIEWIKQRPGHGLEWIGEILPGSGGTNYNEKFKGKATFTAHTSSNTAYMQLSSLTTEDSAIYYCARDSNSFTYWGQGTLVTVSSSGQVLLESNIKVLPTWSTPVQPMALIVLGGVAGLLLFIGLGIFFKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRCVRCRHRRRQAERMSQIKRLLSEKKTCQCPHRFQKTCSPI

SEQ ID NO: 27 - protein, 519 aa, Artificial Sequence
  Synthesized
MALPVTALLLPLALLLHAARPQIVLTQSPAIMSASPGEKVTITCSVSSSVDYIHWFQQKPGTSPKFWIYSTSILASGVPARFSGSGSGTSYSLTISRMEAEDAATYYCQQRSSYPPTFGSGTKLEIKGGGGSGGGGSGGGGSQVQLQQSGGEVMKPGASVKLSCKATGYTFTGYWIEWIKQRPGHGLEWIGEILPGSGGTNYNEKFKGKATFTAHTSSNTAYMQLSSLTTEDSAIYYCARDSNSFTYWGQGTLVTVSSTTTPAPRPPTPAPTIASQPLSLRPEACRPAAGGAVHTRGLDFACDMALIVLGGVAGLLLFIGLGIFFCVRCRHRRRQAERMSQIKRLLSEKKTCQCPHRFQKTCSPIKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 501 aa, Artificial Sequence
  Synthesized
MALPVTALLLPLALLLHAARPQIVLTQSPAIMSASPGEKVTITCSVSSSVDYIHWFQQKPGTSPKFWIYSTSILASGVPARFSGSGSGTSYSLTISRMEAEDAATYYCQQRSSYPPTFGSGTKLEIKGGGGSGGGGSGGGGSQVQLQQSGGEVMKPGASVKLSCKATGYTFTGYWIEWIKQRPGHGLEWIGEILPGSGGTNYNEKFKGKATFTAHTSSNTAYMQLSSLTTEDSAIYYCARDSNSFTYWGQGTLVTVSSTTTPAPRPPTPAPTIASQPLSLRPEACRPAAGGAVHTRGLDFACDIYIWAPLAGTCGVLLLSLVITLYCKKVAKKPTNKAPHPKQEPQEINFPDDLPGSNTAAPVQETLHGCQPVTQEDGKESRISVQERQRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 422 aa, Homo sapiens
HRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 30 - protein, 116 aa, Homo sapiens
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 31 - protein, 93 aa, Homo sapiens
KKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 32 - protein, 72 aa, Homo sapiens
NSRRRCGQKKKLVINSGNGAVEDRKPSGLNGEASKSQEMVHLVNKESSETPDQFMTADETRNLQNVDMKIGV

SEQ ID NO: 33 - protein, 708 aa, Homo sapiens
YKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 34 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 35 - protein, 533 aa, Artificial Sequence
  Synthesized
TTTPAPRPPTPAPTIASQPLSLRPEACRPAAGGAVHTRGLDFACDIYIWAPLAGTCGVLLLSLVITLYCHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGAKRGRKKLLYIFKQPFMRPVQTTQEEDGCSCRFPEEEEGGCEL

SEQ ID NO: 36 - protein, 227 aa, Artificial Sequence
  Synthesized
TTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNKRGRKKLLYIFKQPFMRPVQTTQEEDGCSCRFPEEEEGGCEL

SEQ ID NO: 37 - protein, 151 aa, Artificial Sequence
  Synthesized
TTTPAPRPPTPAPTIASQPLSLRPEACRPAAGGAVHTRGLDFACDIYIWAPLAGTCGVLLLSLVITLYCCVRCRHRRRQAERMSQIKRLLSEKKTCQCPHRFQKTCSPIKRGRKKLLYIFKQPFMRPVQTTQEEDGCSCRFPEEEEGGCEL

SEQ ID NO: 38 - protein, 204 aa, Artificial Sequence
  Synthesized
TTTPAPRPPTPAPTIASQPLSLRPEACRPAAGGAVHTRGLDFACDIYIWAPLAGTCGVLLLSLVITLYCKKLVKKFRQKKQRQWIGPTGMNQNMSFHRNHTATVRSHAENPTASHVDNEYSQPPRNSHLSAYPALEGALHRSSMQPDNSSDSDYDLHGAQRLKRGRKKLLYIFKQPFMRPVQTTQEEDGCSCRFPEEEEGGCEL

SEQ ID NO: 39 - protein, 143 aa, Artificial Sequence
  Synthesized
TTTPAPRPPTPAPTIASQPLSLRPEACRPAAGGAVHTRGLDFACDIYIWAPLAGTCGVLLLSLVITLYCLYCNHRNRRRVCKCPRPVVKSGDKPSLSARYVKRGRKKLLYIFKQPFMRPVQTTQEEDGCSCRFPEEEEGGCEL

SEQ ID NO: 40 - protein, 183 aa, Artificial Sequence
  Synthesized
TTTPAPRPPTPAPTIASQPLSLRPEACRPAAGGAVHTRGLDFACDIYIWAPLAGTCGVLLLSLVITLYCNSRRRCGQKKKLVINSGNGAVEDRKPSGLNGEASKSQEMVHLVNKESSETPDQFMTADETRNLQNVDMKIGVKRGRKKLLYIFKQPFMRPVQTTQEEDGCSCRFPEEEEGGCEL

SEQ ID NO: 41 - protein, 819 aa, Artificial Sequence
  Synthesized
TTTPAPRPPTPAPTIASQPLSLRPEACRPAAGGAVHTRGLDFACDIYIWAPLAGTCGVLLLSLVITLYCYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGSKRGRKKLLYIFKQPFMRPVQTTQEEDGCSCRFPEEEEGGCEL

SEQ ID NO: 42 - protein, 397 aa, Artificial Sequence
  Synthesized
TTTPAPRPPTPAPTIASQPLSLRPEACRPAAGGAVHTRGLDFACDIYIWAPLAGTCGVLLLSLVITLYCNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVKRGRKKLLYIFKQPFMRPVQTTQEEDGCSCRFPEEEEGGCEL

(19) United States
(12) Patent Application Publication
(10) Pub. No.: US 2024/0269182 A1
(43) Pub. Date: Aug. 15, 2024

(54) Modified Chimeric Antigen Receptor and Use thereof

(71) Applicants: Innovative Cellular Therapeutics Holdings, Ltd., George Town, Grand Cayman (KY); Innovative Cellular Therapeutics, Inc., Rockville, MD (US)

(72) Inventors: CHENGFEI PU, Shanghai (CN); WENSHENG WANG, Shanghai (CN); XIAOGANG SHEN, Shanghai (CN); LE TIAN, Rockville, MD (US)

(73) Assignees: Innovative Cellular Therapeutics Holdings, Ltd., George Town, Grand Cayman (KY); Innovative Cellular Therapeutics, Inc., Rockville, MD (US)

(21) Appl. No.: 18/563,764

(22) PCT Filed: May 20, 2022
(86) PCT No.: PCT/US2022/030317

§ 371 Date: Nov. 22, 2023

Related U.S. Application Data
(60) Provisional application No. 63/231,976, filed on Aug. 11, 2021.
(60) Provisional application No. 63/193,837, filed on May 27, 2021.

(51) Int. Cl. A61K 35/17; A61K 39/00; C07K 14/705; C07K 14/725; C07K 14/73; C07K 16/30

(52) U.S. Cl. CPC A61K 35/17; A61K 39/4611; A61K 39/4631; A61K 39/464486; C07K 14/7051; C07K 14/70514; C07K 14/70517; C07K 16/30; A61K 2239/13; A61K 2239/21; A61K 2239/22; A61K 2239/57; C07K 2317/622; C07K 2319/02; C07K 2319/03

(57) ABSTRACT

Embodiments of the present disclosure relate to a polynucleotide encoding a CAR comprising a cytoplasmic domain of CD4, or a CAR comprising SEQ ID NO: 17 in its intracellular domain, and the cytoplasmic domain of CD4 is located between a transmembrane domain of the CAR and a signaling or stimulatory domain, for example, CD3 zeta domain.

### CROSS REFERENCE TO RELATED APPLICATIONS

This application claims the benefit of U.S. Provisional Application 63/193,837, filed May 27, 2021; and U.S. Provisional Application 63/231,976, filed Aug. 11, 2021, which are hereby all incorporated by reference in their entirety.

### SEQUENCE LISTING INFORMATION

A computer readable textfile, entitled “ST25.txt,” created on or about Apr. 6, 2022 with a file size of about 101,568 bytes, contains the sequence listing for this application and is hereby incorporated by reference in its entirety.

### TECHNICAL FIELD

The present disclosure relates to modified cells and uses, in particular to compositions and methods for treating cancer using Chimeric Antigen Receptor (CAR) cells.

### BACKGROUND

Some cancer treatment programs include surgery, radiotherapy, and chemotherapy, targeted therapy and immunotherapy. The drawbacks of these programs include ineffective treatment of advanced cancer, side effects, and patients with poor quality of life. For example, treatment of renal cancer includes resection, targeted therapy (anti-VEGF and mTOR inhibitor, etc.) and immunotherapy (IL-2, PD1 antibody, etc.).

### SUMMARY

Embodiments of the present disclosure relate to a polynucleotide encoding a CAR comprising a cytoplasmic domain of CD4 or a CAR comprising SEQ ID NO: 17 in its intracellular domain, and the cytoplasmic domain of CD4 is located between a transmembrane domain of the CAR and a signaling or stimulatory domain, for example, a CD3 zeta domain. In embodiments, the CAR comprises the SEQ ID NO: 18 and a scFv targeting a solid tumor antigen.

This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 shows a schematic diagram of an exemplary CAR structure.

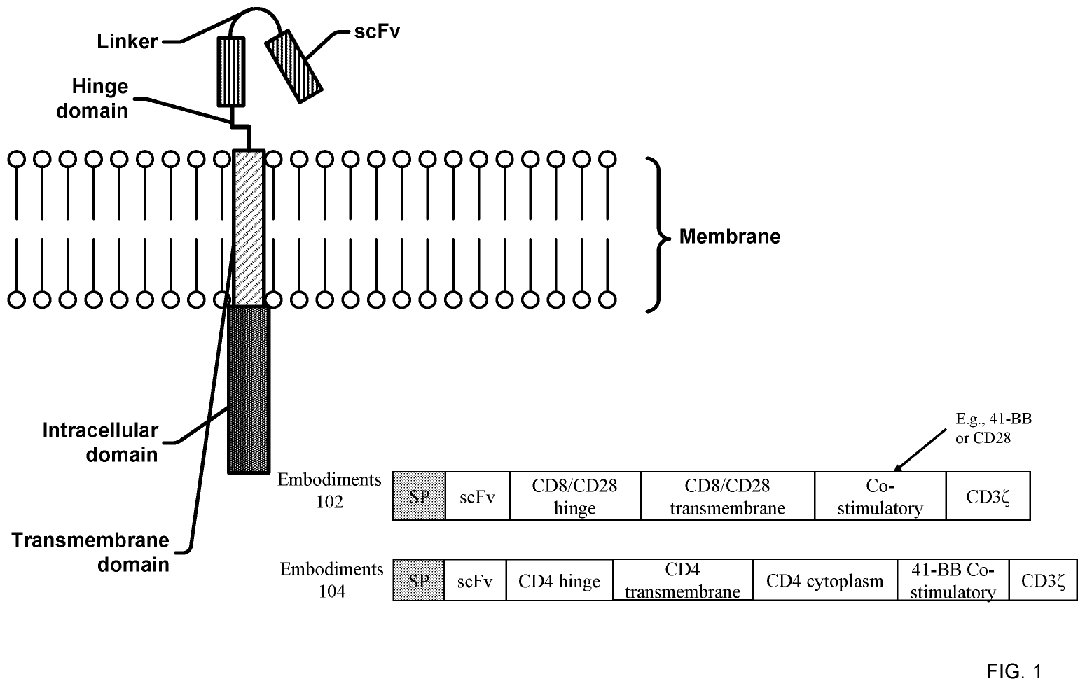

FIG. 2 shows exemplary structures of various vectors.

FIG. 3 shows a schematic diagram of exemplary cell membrane proteins, including a conventional CAR and a fusion protein.

FIG. 4 shows a schematic diagram of a cell membrane protein, including a conventional CAR.

FIG. 5 shows exemplary structures of various vectors. “SP” refers to “signal peptide”, and “P2A” is a 18-22 amino acid long peptide that mediates cleavage of peptides during translation in eukaryotic cells.

FIG. 6 shows exemplary structures of various vectors.

FIG. 7 shows exemplary structures of various vectors.

FIG. 8 shows exemplary structures of various vectors.

FIG. 9 shows exemplary structures of various vectors.

FIGS. 10A, 10B, 10C show the anti-tumor effect of CAR T cells. FIG. 10A shows the expression of CAR in human embryonic kidney (HEK) cell line 293T and human tumor cell line A375 with Western Blot. FIG. 10B shows the expression of CAR in cell line 293T and A375 with Immunofluorescence (IF) staining. FIG. 10C shows expression of CD137 in CAR-T cells co-cultured with the A375 cell lines.

FIG. 11 shows flow cytometry results of the expression of CAR with and without CD4 modification and CD40L on T cells that were co-cultured with substrates cells.

FIGS. 12A, 12B, and 12C show cytokine (IL2 and TNF-α) release and cell expansion of CAR T cells co-cultured with substrate cells.

FIG. 13 shows an exemplary CAR comprising a LCK binding domain for binding LCK which binds a co-receptor.

FIG. 14 shows flow cytometry results of various CARs expressed on T cells.

FIGS. 15A and 15B show expression of MAGE-A4 on A375 cells.

FIGS. 16A and 16B show flow cytometry results of the expansion of CAR T cells co-cultured with A375 cells for 96 hours.

FIG. 17 shows flow cytometry results of the expression of MAGE-A4 in A375 and A375-P cells.

FIGS. 18A, 18B, 18C, and 18D show flow cytometry results of cell expansion of various CAR T cells co-cultured with A375 (FIG. 18A) cells or A375-P cells (FIG. 18B).

### DETAILED DESCRIPTION

Unless defined otherwise, all technical and scientific terms used herein have the same meaning as commonly understood by those of ordinary skill in the art to which the disclosure belongs. Although any method and material similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and refers to monoclonal antibodies (including full length monoclonal antibodies), polyclonal antibodies, multi-specific antibodies (e.g., bispecific antibodies), and antibody fragments so long as they exhibit the desired biological activity or function. The antibodies in the present disclosure may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragments” refers to a portion of a full length antibody, for example, the antigen binding or variable region of the antibody. Other examples of antibody fragments include Fab, Fab′, F(ab′)2, and Fv fragments; diabodies; linear antibodies; single-chain antibody molecules; and multi-specific antibodies formed from antibody fragments.

The term “Fv” refers to the minimum antibody fragment which contains a complete antigen-recognition and -binding site. This fragment consists of a dimer of one heavy- and one light-chain variable region domain in tight, non-covalent association. From the folding of these two domains emanates six hypervariable loops (3 loops each from the H and L chain) that contribute the amino acid residues for antigen binding and confer antigen binding specificity to the antibody. However, even a single variable domain (or half of a Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

The term “synthetic antibody” refers to an antibody which is generated using recombinant DNA technology, such as, for example, an antibody expressed by a bacteriophage. The term also includes an antibody which has been generated by the synthesis of a DNA molecule encoding the antibody and the expression of the DNA molecule to obtain the antibody, or to obtain an amino acid encoding the antibody. The synthetic DNA is obtained using technology that is available and well known in the art.

The term “antigen” refers to a molecule that provokes an immune response, which may involve either antibody production, or the activation of specific immunologically-competent cells, or both. Antigens include any macromolecule, including all proteins or peptides, or molecules derived from recombinant or genomic DNA. For example, DNA including a nucleotide sequence or a partial nucleotide sequence encoding a protein or peptide that elicits an immune response, and therefore, encodes an “antigen” as the term is used herein. An antigen need not be encoded solely by a full-length nucleotide sequence of a gene. An antigen can be generated, synthesized or derived from a biological sample including a tissue sample, a tumor sample, a cell, or a biological fluid.

The term “anti-tumor effect” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies in the prevention of the occurrence of tumor in the first place.

The term “auto-antigen” refers to an antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject which is subsequently re-introduced into the same subject.

The term “allogeneic” is used to describe a graft derived from a different subject of the same species. As an example, a donor subject may be a related or unrelated or recipient subject, but the donor subject has immune system markers which are similar to the recipient subject.

The term “xenogeneic” is used to describe a graft derived from a subject of a different species. As an example, the donor subject is from a different species than a recipient subject and the donor subject and the recipient subject can be genetically and immunologically incompatible.

The term “cancer” refers to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Cancers that may be treated include tumors that are not vascularized, or not yet substantially vascularized, as well as vascularized tumors. The cancers may include non-solid tumors (such as hematological tumors, for example, leukemias and lymphomas) or may include solid tumors. Types of cancers to be treated with the CARs of the disclosure include, but are not limited to, carcinoma, blastoma, and sarcoma, and certain leukemia or lymphoid malignancies, benign and malignant tumors, and malignancies, e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastases).

A solid tumor antigen is an antigen expressed on a solid tumor. In embodiments, solid tumor antigens are also expressed at low levels on healthy tissue. Examples of solid tumor antigens and their related disease tumors are provided in Table 1.

TABLE 1

| Solid Tumor | |
|---|---|
| antigen | Disease tumor |
| PRLR | Breast Cancer |
| CLCA1 | colorectal Cancer |
| MUC12 | colorectal Cancer |
| GUCY2C | colorectal Cancer |
| GPR35 | colorectal Cancer |
| CR1L | Gastric Cancer |
| MUC 17 | Gastric Cancer |
| TMPRSS11B | esophageal Cancer |
| MUC21 | esophageal Cancer |
| TMPRSS11E | esophageal Cancer |
| CD207 | bladder Cancer |
| SLC30A8 | pancreatic Cancer |
| CFC1 | pancreatic Cancer |
| SLC12A3 | Cervical Cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/Urothelial cancer |
| UPK2 | Urothelial cancer (including bladder cancer) |
| ADAM12 | Breast cancer, pancreatic cancer and the like |
| SLC45A3 | Prostate cancer |
| ACPP | Prostate cancer |
| MUC21 | Esophageal cancer |
| MUC16 | Ovarian cancer |
| MS4A12 | Colorectal cancer |
| ALPP | Endometrial cancer |
| CEA | Colorectal carcinoma |
| EphA2 | Glioma |
| FAP | Mesothelioma |
| GPC3 | Lung squamous cell carcinoma |
| IL13-Rα2 | Glioma |
| Mesothelin | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | Carcinomas |
| EpCAM | Carcinomas |
| EGFRvIII | Glioma-Glioblastoma |
| EGFR | Glioma-NSCL cancer |
| tMUC 1 | Cholangiocarcinoma, Pancreatic cancer, Breast Cancer |
| PSCA | pancreas, stomach, or prostate cancer |

Throughout this specification, unless the context requires otherwise, the words “comprise,” “includes” and “including” will be understood to imply the inclusion of a stated step or element (ingredients or components) or group of steps or elements (ingredients or components) but not the exclusion of any other step or element or group of steps or elements.

The phrase “consisting of” is meant to include, and is limited to, whatever follows the phrase “consisting of.” Thus, the phrase “consisting of” indicates that the listed elements or steps are required or mandatory and that no other elements may be present.

The phrase “consisting essentially of” is meant to include any element listed after the phrase and can include other elements or steps that do not interfere with or contribute to the activity or action specified in the disclosure for the listed elements. Thus, the phrase “consisting essentially of” indicates that the listed elements or steps are required or mandatory, but that other elements or steps are optional and may or may not be present depending upon whether or not they affect the activity or action of the listed elements or steps. In embodiments, those elements or steps that do not affect an embodiment are those elements or steps that do not alter the embodiment's ability in a statistically significant manner to perform a function in vitro or in vivo, such as killing cancer cells in vitro or in vivo.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

The term “co-stimulatory ligand” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand can include B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor and a ligand that specifically binds with B7-H3. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal, which in combination with a primary signal, such as TCR/CD3 ligation, leads to T cell proliferation and/or upregulation or downregulation of key molecules.

The terms “co-stimulatory signaling region”, “co-stimulatory domain”, and “co-stimulation domain” are used interchangeably to refer to one or more additional stimulatory domain in addition to a stimulatory or signaling domain such as CD3 zeta. The terms “stimulatory” or “signaling” domain (or region) are also used interchangeably, when referring to CD3 zeta.

The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis, and wherein if the disease is not ameliorated then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally-occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression” refers to the transcription and/or translation of a particular nucleotide sequence driven by its promoter.

The term “expression vector” refers to a vector including a recombinant polynucleotide including expression control sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. A comparison is made when two sequences are aligned to give maximum homology.

The term “immunoglobulin” or “Ig,” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule such as a protein or nucleic acid. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The term “substantially purified” refers to a material that is substantially frr from components that normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables integration of the genetic information into the host chromosome resulting in stably transduced genetic information. HIV, SIV, and FIV are all examples of lentiviruses. Vectors derived from lentiviruses offer the means to achieve significant levels of gene transfer in vivo.

The term “modulating,” refers to mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

The term “under transcriptional control” refers to a promoter being operably linked to and in the correct location and orientation in relation to a polynucleotide to control the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

The term “parenteral administration” of a composition includes, e.g., subcutaneous (s.c.), intravenous (i.v.), intramuscular (i.m.), intrasternal injection, or infusion techniques.

The terms “patient,” “subject,” and “individual,” and the like are used interchangeably herein, and refer to any human, animal, or living organism, amenable to the methods described herein. In certain non-limiting embodiments, the patient, subject, or individual is a human or animal. In embodiments, the term “subject” is intended to include living organisms in which an immune response can be elicited (e.g., mammals). Examples of subjects include humans, and animals such as dogs, cats, mice, rats, and transgenic species thereof.

A subject in need of treatment or in need thereof includes a subject having a disease, condition, or disorder that needs to be treated. A subject in need thereof also includes a subject that needs treatment for prevention of a disease, condition, or disorder. In embodiments, the disease is cancer.

The term “polynucleotide” or “nucleic acid” refers to mRNA, RNA, CRNA, rRNA, cDNA or DNA. The term typically refers to a polymeric form of nucleotides of at least 10 bases in length, either ribonucleotides or deoxynucleotides or a modified form of either type of nucleotide. The term includes all forms of nucleic acids including single and double stranded forms of nucleic acids.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In embodiments, a polypeptide variant is distinguished from a reference polypeptide by one or more substitutions, which may be conservative or non-conservative. In embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted or replaced with different amino acid residues.

The term “promoter” refers to a DNA sequence recognized by the synthetic machinery of the cell or introduced synthetic machinery, required to initiate the specific transcription of a polynucleotide sequence. The term “expression control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The term “bind,” “binds,” or “interacts with” refers to a molecule recognizing and adhering to a second molecule in a sample or organism but does not substantially recognize or adhere to other structurally unrelated molecules in the sample. The term “specifically binds,” as used herein with respect to an antibody, refers to an antibody which recognizes a specific antigen, but does not substantially recognize or bind other molecules in a sample. For example, an antibody that specifically binds an antigen from one species may also bind that antigen from one or more species. But, such cross-species reactivity does not itself alter the classification of an antibody as specific. In another example, an antibody that specifically binds an antigen may also bind different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody, will reduce the amount of labeled A bound to the antibody.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding, and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

Zinc finger binding domains can be “engineered” to bind to a predetermined nucleotide sequence, for example via engineering (altering one or more amino acids) of the recognition helix region of a naturally occurring zinc finger protein. Further, a Zinc finger binding domain may be fused a DNA-cleavage domain to form a Zinc finger nuclease (ZFN) targeting a specific desired DNA sequence. For example, a pair of ZFNs (e.g., a ZFN-left arm and a ZFN-right arm) may be engineered to target and cause modifications of specific desired DNA sequences (e.g., TRAC genes).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In embodiments, fusion polypeptides are used for targeted double-stranded DNA cleavage.

A “target site” or “target sequence” is a nucleic acid sequence that defines a portion of a nucleic acid to which a binding molecule will bind, provided sufficient conditions for binding exist. For example, the sequence 5′ GAATTC 3′ is a target site for the Eco RI restriction endonuclease.

A “fusion” molecule is a molecule in which two or more subunit molecules are linked, preferably covalently. The subunit molecules can be the same chemical type of molecule or can be different chemical types of molecules. Examples of the first type of fusion molecule include, but are not limited to, fusion proteins (for example, a fusion between a ZFP DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage, and polypeptide ligation can also be involved in the expression of the protein in a cell. Methods for polynucleotide and polypeptide delivery to cells are presented elsewhere in this disclosure.

“Modulation” of gene expression refers to a change in the activity of a gene. Modulation of expression can include but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

By “statistically significant,” it is meant that the result was unlikely to have occurred by chance. Statistical significance can be determined by any method known in the art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The term “stimulation,” refers to a primary response induced by binding of a stimulatory molecule (e.g., a TCR/CD3 complex) with its cognate ligand thereby mediating a signal transduction event, such as signal transduction via the TCR/CD3 complex. Stimulation can mediate altered expression of certain molecules, such as downregulation of TGF-β, and/or reorganization of cytoskeletal structures. CD3 zeta is not the only suitable primary signaling domain for a CAR construct with respect to the primary response. For example, back in 1993, both CD3 zeta and FcR gamma were shown as functional primary signaling domains of CAR molecules. Eshhar et al., “Specific activation and targeting of cytotoxic lymphocytes through chimeric single chains consisting of antibody-binding domains and the gamma or zeta subunits of the immunoglobulin and T cell receptors” PNAS, 1993 Jan. 15; 90(2):720-4, showed that two CAR constructs in which an scFv was fused to “either the FcR gamma chain or the CD3 complex chain” triggered T cell activation and target cell. Notably, as demonstrated in Eshhar et al., CAR constructs containing only the primary signaling domain CD3 zeta or FcR gamma are functional without the co-presence of co-stimulatory domains. Additional non-CD3 zeta based CAR constructs have been developed over the years. For example, Wang et al. (“A Chimeric Antigen Receptor (CARs) Based Upon a Killer Immunoglobulin-Like Receptor (KIR) Triggers Robust Cytotoxic Activity in Solid Tumors” Molecular Therapy, vol. 22, no. Suppl. 1, May 2014, page S57) tested a CAR molecule in which an scFv was fused to “the transmembrane and cytoplasmic domain of a killer immunoglobulin-like receptor (KIR). Wang et al. reported that, “a KIR-based CAR targeting mesothelin (SS 1-KIR) triggers antigen-specific cytotoxic activity and cytokine production that is comparable to CD3˜-based CARs.” A second publication from the same group, Wang et al. (“Generation of Potent T-cell Immunotherapy for Cancer Using DAP12-Based, Multichain, Chimeric Immunoreceptors” Cancer Immunol Res. 2015 July; 3(7):815-26) showed that a CAR molecule in which “a single-chain variable fragment for antigen recognition was fused to the transmembrane and cytoplasmic domains of KIR2DS2, a stimulatory killer immunoglobulin-like receptor (KIR)” functioned both in vitro and in vivo “when introduced into human T cells with DAP12, an immunotyrosine-based activation motifs-containing adaptor.”

The term “stimulatory molecule” refers to a molecule on a T cell that specifically binds a cognate stimulatory ligand present on an antigen presenting cell. For example, a functional signaling domain derived from a stimulatory molecule is the zeta chain associated with the T cell receptor complex.

The term “stimulatory ligand” refers to a ligand that when present on an antigen presenting cell (e.g., an APC, a dendritic cell, a B-cell, and the like.) can specifically bind with a cognate binding partner (referred to herein as a “stimulatory molecule”) on a cell, for example a T cell, thereby mediating a primary response by the T cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to a treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject that is being sought by the researcher, veterinarian, medical doctor or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease and its severity and the age, weight, etc., of the subject to be treated.

The term “treat a disease” refers to the reduction of the frequency or severity of at least one sign or symptom of a disease or disorder experienced by a subject.

The term “transfected” or “transformed” or “transduced” refers to a process by which an exogenous nucleic acid is transferred or introduced into the host cell. A “transfected” or “transformed” or “transduced” cell is one which has been transfected, transformed, or transduced with exogenous nucleic acid. The cell includes the primary subject cell and its progeny.

The term “vector” refers to a polynucleotide that comprises an isolated nucleic acid and which can be used to deliver the isolated nucleic acid to the interior of a cell in vitro and in vivo (in a subject). Numerous vectors are known in the art including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds which facilitate transfer of nucleic acid into cells, such as, for example, polylysine compounds, liposomes, and the like. Examples of viral vectors include, adenoviral vectors, adeno-associated virus vectors, retroviral vectors, and others. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted making the vector biologically safe.

Embodiments of the present disclosure relate to a vector comprising the polynucleotide described herein, a cell comprising the polynucleotide, a pharmaceutical composition comprising a population of the cell, and a method or use of the polynucleotide. For example, the method comprises providing a viral particle (e.g., AAV, lentivirus or their variants) comprising a vector genome, the vector genome comprising the polynucleotide and a polynucleotide encoding an antigen binding molecule, the polynucleotide operably linked to an expression control element conferring transcription of the polynucleotides; and administering an amount of the viral particle to a subject such that the polynucleotide is expressed in the subject, where the one or more molecules are overexpressed in cancer cells, associated with recruitment of immune cells, and/or associated with autoimmunity. In embodiments, the AAV preparation may include AAV vector particles, empty capsids and host cell impurities, thereby providing an AAV product substantially free of AAV empty capsids.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “chimeric antigen receptor” or alternatively a “CAR” refers to a recombinant polypeptide construct comprising at least an extracellular antigen binding domain, a transmembrane domain and an intracellular signaling domain (e.g., cytoplasmic domain). In embodiments, the domains in the CAR polypeptide construct are on the same polypeptide chain (e.g., comprising a chimeric fusion protein). In embodiments, the domains of the CAR polypeptide are not on the same molecule, e.g. not contiguous with each other or are on different polypeptide chains.

In embodiments, the intracellular signaling domain may include a functional signaling domain derived from a stimulatory molecule and/or a co-stimulatory molecule as described herein. In embodiments, the intracellular signaling domain includes a functional signaling domain derived from a primary signaling domain (e.g., a primary signaling domain of CD3-zeta). In embodiments, the intracellular signaling domain further includes one or more functional signaling domains derived from at least one co-stimulatory molecule. The co-stimulatory signaling region refers to a portion of the CAR including the intracellular domain of a co-stimulatory molecule. Co-stimulatory molecules can include cell surface molecules for inducing an efficient response from the lymphocytes (in response to an antigen).

Between the extracellular domain and the transmembrane domain of the CAR, there may be incorporated a spacer domain. As used herein, the term “spacer domain” generally means any oligo- or polypeptide that functions to link the transmembrane domain to the extracellular domain and/or the cytoplasmic domain in the polypeptide chain. A spacer domain may include up to 300 amino acids, 10 to 100 amino acids, or 25 to 50 amino acids.

The extracellular domain of a CAR may include an antigen binding domain (e.g., a scFv, a single domain antibody, or TCR, such as a TCR alpha binding domain or a TCR beta binding domain) that targets a specific tumor marker (e.g., a tumor antigen). Tumor antigens are proteins that are produced by tumor cells that elicit an immune response, particularly T cell mediated immune responses. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. For example, when the antigen that the CAR binds is CD19, the CAR thereof is referred to as CD19CAR.

In embodiments, the extracellular ligand-binding domain comprises a scFv comprising the light chain variable (VL) region and the heavy chain variable (VH) region of a target antigen-specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)3 (SEQ ID: 50), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides comprising about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In embodiments, the tumor antigen includes HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor α 2, IL-11 receptor α, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, or viral-associated antigens expressed by a tumor. In embodiments, the binding element of the CAR may include any antigen binding moiety that when bound to its cognate antigen, affects a tumor cell such that the tumor cell fails to grow, or is promoted to die or diminish.

The present disclosure also relates to a bispecific chimeric antigen receptor, a polynucleotide encoding the bispecific chimeric antigen receptor, and/or a modified cell comprising the polynucleotide, wherein the bispecific chimeric antigen receptor comprises a first antigen binding domain, a second antigen binding domain, a cytoplasmic domain, and a transmembrane domain, and wherein the first antigen binding domain recognizes a first antigen, and the second antigen binding domain recognize a second antigen. In embodiments, the first antigen is an antigen associated with a white blood cell, and the second antigen is a solid tumor antigen. In embodiments, the first and second antigens are identical or different. In embodiments, the first and second antigens are both solid tumor antigens.

In embodiments, the intracellular domain of the CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

In embodiments, the intracellular domain comprises a CD3 zeta signaling domain. Embodiments relate to a vector comprising the isolated nucleic acid sequence described herein. Embodiments relate to an isolated cell comprising the isolated nucleic acid sequence described herein.

Embodiments relate to a composition comprising a population of cells including T cells comprising the CAR described herein. Embodiments relate to a CAR encoded by the isolated nucleic acid sequence described herein. In embodiments, an isolated nucleic acid sequence encodes a chimeric antigen receptor (CAR), wherein the CAR comprises an extracellular domain, a transmembrane domain, and an intracellular domain, wherein the extracellular domain binds an antigen of a tumor (e.g., solid tumor). In embodiments, the extracellular domain binds MSLN, GPC-3, CD205, ALPP, and CD70. In embodiments, the extracellular domain binds human MSLN, GPC-3, CD205, ALPP, and CD70. In embodiments, the CAR comprises one of the amino acid sequences of SEQ in Table 2. In embodiments, a method of eliciting and/or enhancing T− cell response in a subject having a solid tumor or treating a solid tumor in the subject, the method comprising administering an effective amount of T cells comprising the CAR.

Mesothelin is a glycosylphosphatidylinositol-anchored cell-surface protein that may function as a cell adhesion protein. This protein is overexpressed in epithelial mesotheliomas, ovarian cancers and in specific squamous cell carcinomas. ALPP is a metalloenzyme that catalyzes the hydrolysis of phosphoric acid monoesters. The protein is primarily expressed in placental and endometrial tissue; however, strong ectopic expression has been detected in ovarian adenocarcinoma, serous cystadenocarcinoma, and other ovarian cancer cells. GPC-3 is a member of the glypican-related integral membrane proteoglycan family (GRIPS) contain a core protein anchored to the cytoplasmic membrane via a glycosyl phosphatidylinositol linkage. These proteins may play a role in the control of cell division and growth regulation. The protein encoded by this gene can bind to and inhibit the dipeptidyl peptidase activity of CD26, and it can induce apoptosis in certain cell types. CD70 is a cytokine belongs to the tumor necrosis factor (TNF) ligand family. This cytokine is a ligand for TNFRSF27/CD27. It is a surface antigen on activated, but not on resting, T and B lymphocytes. It induces proliferation of co-stimulated T cells, enhances the generation of cytolytic T cells, and contributes to T cell activation. This cytokine is also reported to play a role in regulating B-cell activation, cytotoxic function of natural killer cells, and immunoglobulin synthesis. Examples of CD70 CAR include 1) a traditional carrier (41-BB and CD3 zeta) followed by a CD70 scFv, 2) a traditional carrier connected to the extracellular segment of CD27, and 3) CD27 FL C-terminal integrated with a CD3 zeta (e.g., CD27 and CD3 zeta).

CD205 is a type I endocytic receptor protein to direct captured antigens from the extracellular space to a specialized antigen-processing compartment.

The cells, including CAR cells and modified cells, described herein can be derived from a stem cell. The stem cells may be adult stem cells, embryonic stem cells, or non-human stem cells, cord blood stem cells, progenitor cells, bone marrow stem cells, induced pluripotent stem cells, totipotent stem cells, or hematopoietic stem cells. The cells can also be a dendritic cell, a NK-cell, a B-cell, or a T cell selected from the group consisting of inflammatory T lymphocytes, cytotoxic T lymphocytes, regulatory T lymphocytes, and helper T lymphocytes. In embodiments, the cells can be derived from the group consisting of CD4+T-lymphocytes and CD8+T-lymphocytes. Prior to expansion and genetic modification of the cells described herein, a source of cells may be obtained from a subject through a variety of non-limiting methods. T cells may be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In embodiments, any number of T cell lines available and known to those skilled in the art, can be used. In embodiments, the cells may be derived from a healthy donor, from a patient diagnosed with cancer or from a patient diagnosed with an infection. In embodiments, the cells are part of a mixed population of cells which present different phenotypic characteristics.

T cells, or T lymphocytes, are a type of white blood cell of the immune system. There are various types of T cells including T helper (T_{H}) cells, cytotoxic T (T_{C}) cells (T killer cells, killer T cells), natural killer T (NKT) cells, memory T (Tm) cells, regulatory T (Treg) cells, and gamma delta T (γδ T) cells.

T helper (T_{H}) cells assist other lymphocytes, for example, activating cytotoxic T cells and macrophages and maturation of B cells into plasma cells and memory B cells. These T helper cells express CD4 glycoprotein on their surface and are also known as CD4⁺ T cells. Once activated, these T cells divide rapidly and secrete cytokines.

Cytotoxic T (T_{C}) cells destroy virus-infected cells and tumor cells and are also involved in transplant rejection. They express CD8 protein on their surface. Cytotoxic T cell release cytokines.

Natural Killer T (NKT) cells are different from natural killer cells. NKT cells recognize glycolipid antigens presented by CD1d. Once activated, NKT cells produce cytokine and release cell killing molecules.

Memory T (Tm) cells are long-lived and can expand to large number of effector T cells upon re-exposure to their cognate antigen. Tm cells provide the immune system with memory against previously encountered pathogens. There are various subtypes of Tm cells including central memory T (T_{CM}) cells, effector memory T (T_{EM}) cells, tissue resident memory T (T_{RM}) cells, and virtual memory T cells. Tm cells are either CD4⁺ or CD8⁺ and usually CD45RO.

Regulatory T (Treg) cells shut down T cell mediated immunity at the end of an immune reaction and suppress autoreactive T cells that escaped the process of negative selection in the thymus. Subsets of Treg cells include thymic Treg and peripherally derived Treg. Both subsets of Treg require the expression of the transcription factor FOXP3.

Gamma delta T (γδ T) cells are a subset of T cells that possess a γδ T cell receptor (TCR) on the cell surface, as most T cells express the αβ TCR chains. γδ T cells are less common in human and mice and are mainly found in the gut mucosa, skin, lung, and uterus. They are involved in the initiation and propagation of immune responses.

The term “stem cell” refers to any type of cell which has the capacity for self-renewal and the ability to differentiate into other kind(s) of cell. For example, a stem cell gives rise either to two daughter stem cells (as occurs in vitro with embryonic stem cells in culture) or to one stem cell and a cell that undergoes differentiation (as occurs e.g. in hematopoietic stem cells, which give rise to blood cells). Different categories of stem cells may be distinguished on the basis of their origin and/or on the extent of their capacity for differentiation into other types of cell. Stem cells can include embryonic stem (ES) cells (i.e., pluripotent stem cells), somatic stem cells, induced pluripotent stem cells, and any other types stem cells.

Pluripotent embryonic stem cells can be found in the inner cell mass of a blastocyst and have high innate capacity for differentiation. For example, pluripotent embryonic stem cells have the potential to form any type of cell in the body. When grown in vitro for long periods of time, ES cells maintain pluripotency, and progeny cells retain the potential for multilineage differentiation.

Somatic stem cells can include fetal stem cells (from the fetus) and adult stem cells (found in various tissues, such as bone marrow). These cells have been regarded as having a capacity for differentiation lower than that of the pluripotent ES cells—with the capacity of fetal stem cells being greater than that of adult stem cells; they apparently differentiate into only a limited number of different types of cells and have been described as multipotent. “Tissue-specific” stem cells normally give rise to only one type of cell. For example, embryonic stem cells can differentiate into blood stem cells (e.g., Hematopoietic stem cells (HSCs)), which can further differentiate into various blood cells (e.g., red blood cells, platelets, white blood cells, etc.).

Induced pluripotent stem cells (iPS cells or iPSCs) can include a type of pluripotent stem cell artificially derived from a non-pluripotent cell (e.g., an adult somatic cell) by inducing expression of specific genes. Induced pluripotent stem cells are similar to naturally occurring pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells can be isolated from adult stomach, liver, skin cells, and blood cells.

In embodiments, the CAR cells, the modified cell, or the cell is a T cell, a NK cell, a macrophage or a dendritic cell. For example, the CAR cells, the modified cell, or the cell is a T cell.

In embodiments, the antigen binding molecule is a T Cell Receptor (TCR). In embodiments, the TCR is modified TCR. In embodiments, the TCR is derived from spontaneously occurring tumor-specific T cells in patients. In embodiments, the TCR binds a tumor antigen. In embodiments, the tumor antigen comprises CEA, gp100, MART-1, p53, MAGE-A3, or NY-ESO-1. In embodiments, the TCR comprises TCRγ and TCRδ chains or TCRα and TCRβ chains. In embodiments, a T cell clone that expresses a TCR with high affinity for the target antigen may be isolated. In embodiments, tumor-infiltrating lymphocytes (TILs) or peripheral blood mononuclear cells (PBMCs) may be cultured in the presence of antigen-presenting cells (APCs) pulsed with a peptide representing an epitope known to elicit a dominant T cell response when presented in the context of a defined HLA allele. High-affinity clones may be then selected on the basis of MHC-peptide tetramer staining and/or the ability to recognize and lyse target cells pulsed with low titrated concentrations of cognate peptide antigen. After the clone has been selected, the TCRα and TCRβ chains or TCRγ and TCRδ chains are identified and isolated by molecular cloning. For example, for TCRα and TCRβ chains, the TCRα and TCRβ gene sequences are then used to generate an expression construct that ideally promotes stable, high-level expression of both TCR chains in human T cells. The transduction vehicle (e.g., a gammaretrovirus or lentivirus) may be then generated and tested for functionality (antigen specificity and functional avidity) and used to produce a clinical lot of the vector. An aliquot of the final product is then used to transduce the target T cell population (generally purified from patient PBMCs), which is expanded before infusion into the subject.

In embodiments, the binding element of the CAR may include any antigen binding moiety that when bound to its cognate antigen, affects a tumor cell for example, it kills the tumor cell, inhibits the growth of the tumor cell, or promotes death of the tumor cell.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The embodiments of the present disclosure further relate to vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Viruses can be used to deliver nucleic acids into a cell in vitro and in vivo (in a subject). Examples of viruses useful for delivery of nucleic acids into cells include retrovirus, adenovirus, herpes simplex virus, vaccinia virus, and adeno-associated virus.

There also exist non-viral methods for delivering nucleic acids into a cell, for example, electroporation, gene gun, sonoporation, magnetofection, and the use of oligonucleotides, lipoplexes, dendrimers, and inorganic nanoparticles.

The expression of natural or synthetic nucleic acids encoding CARs is typically achieved by operably linking a nucleic acid encoding the CAR polypeptide or portions thereof to one or more promoters and incorporating the construct into an expression vector. The vectors can be suitable for replication and integration into eukaryotes. Typical cloning vectors contain transcription and translation terminators, initiation sequences, and promoters useful for regulation of the expression of the desired nucleic acid sequence.

Additional information related to expression of synthetic nucleic acids encoding CARs and gene transfer into mammalian cells is provided in U.S. Pat. No. 8,906,682, incorporated by reference in its entirety.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. In embodiments, activated T cells are administered to a subject and then subsequently blood is redrawn (or have apheresis performed). T cells are collected, expanded, and reinfused into the subject. This process can be carried out multiple times every few weeks. In embodiments, T cells can be activated from blood draws of from 10 cc to 400 cc. In embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocols, certain populations of T cells may be selected.

The administration of the pharmaceutical compositions described herein may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i. v.) injection, or intraperitoneally. In embodiments, the T cell compositions of the present disclosure are administered to a patient by intradermal or subcutaneous injection. In embodiments, the T cell compositions of the present disclosure are preferably administered by i.v. injection. The compositions of T cells may be injected directly into a tumor, lymph node, or site of infection. In embodiments of the present disclosure, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the present disclosure may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In embodiments, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In embodiments, the cell compositions of the present disclosure are administered following B-cell ablative therapy such as agents that react with CD20, e.g., Rituxan. For example, subjects may undergo standard treatment with high dose chemotherapy followed by peripheral blood stem cell transplantation. In embodiments, following the transplant, subjects receive an infusion of the expanded immune cells of the present disclosure. In embodiments, expanded cells are administered before or following surgery.

The dosage of the above treatments to be administered to a patient will vary with the precise nature of the condition being treated and the recipient of the treatment. The scaling of dosages for human administration can be performed according to art-accepted practices by a physician depending on various factors.

Additional information on the methods of cancer treatment using engineered or modified T cells is provided in U.S. Pat. No. 8,906,682, incorporated by reference in its entirety.

In embodiments, the population of cells described herein is used in autologous CAR T cell therapy. In embodiments, the CAR T cell therapy is allogenic CAR T cell therapy, TCR T cell therapy, and NK cell therapy.

Embodiments relate to an in vitro method for preparing modified cells. The method may include obtaining a sample of cells from the subject. For example, the sample may include T cells or T cell progenitors. The method may further include transfecting the cells with a DNA encoding at least a CAR, culturing the population of CAR cells ex vivo in a medium that selectively enhances proliferation of CAR-expressing T cells.

In embodiments, the sample is a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T cells.

Embodiments of the present disclosure relate to treating cancer using Chimeric Antigen Receptor (CAR) cells using a molecule associated with a gene fusion. Embodiments relate to an isolated nucleic acid sequence encoding a chimeric antigen receptor (CAR), wherein the CAR comprises an extracellular domain, a transmembrane domain, and an intracellular domain, wherein the extracellular domain binds a gene fusion antigen of a gene fusion.

As used herein, the term “gene fusion” refers to the fusion of at least a portion of a gene to at least a portion of an additional gene. The gene fusion need not include entire genes or exons of genes. In some instances, gene fusion is associated with alternations in cancer. A gene fusion product refers to a chimeric genomic DNA, a chimeric messenger RNA, a truncated protein or a chimeric protein resulting from a gene fusion. The gene fusion product may be detected by various methods described in U.S. Pat. No. 9,938,582, which is incorporated as a reference herein. A “gene fusion antigen” refers to a truncated protein or a chimeric protein that results from a gene fusion. In embodiments, an epitope of a gene fusion antigen may include a part of the gene fusion antigen or an immunogenic part of another antigen caused by the gene fusion. In embodiments, the gene fusion antigen interacts with, or is part of, cell membranes.

In embodiments, detection of mRNA and protein expression levels of the target molecules (listed in Table 2) in human cells may be performed using experimental methods such as qPCR and FACS. Further, target molecules specifically expressed in the corresponding tumor cells with very low expression or undetectable expression in normal tissue cells may be identified.

In embodiments, In Vitro Killer Assay as well as killing experiment of CAR T Cells Co-Cultured with Antigen-Positive Cells may be performed. CAR T cells may exhibit a killing effect on the corresponding antigen-positive cells, a decrease in the number of corresponding antigen-positive cells co-cultured with CAR T cells, and an increase in the release of IFNγ, TNFα, etc. as compared to control cells that did not express the corresponding antigen.

In embodiments, In Vivo Killer Assay may be performed. For example, mice may be transplanted with corresponding antigen tumor cells, and tumorigenic, transfusion of CAR T cells, and a decrease in mouse tumors and mouse blood IFNγ, TNFα, and other signals may be defected.

Embodiments relate to a method of eliciting and/or enhancing T cell response in a subject having a solid tumor or treating a solid tumor in the subject, the method comprising administering an effective amount of T cells comprising the CAR described herein. In embodiments, the intracellular domain of the CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. In embodiments, the intracellular domain comprises a CD3 zeta signaling domain.

Embodiments relate to a vector comprising the isolated nucleic acid described herein.

Embodiments relate to an isolated cell comprising the isolated nucleic acid sequence described herein. Embodiments relate to a composition comprising a population of T cells comprising the CAR described herein. Embodiments relate to a CAR encoded by the isolated nucleic acid sequence described herein. Embodiments relate to a method of eliciting and/or enhancing T− cell response in a subject or treating a tumor of the subject, the method comprising: administering an effective amount of T cell comprising the CAR described herein.

In embodiments, the CAR molecules described herein comprise one or more complementarity-determining regions (CDRs) for binding an antigen of interest. CDRs are part of the variable domains in immunoglobulins and T cell receptors for binding a specific antigen. There are three CDRs for each variable domain. Since there is a variable heavy domain and a variable light domain, there are six CDRs for binding an antigen. Further since an antibody has two heavy chains and two light chains, an antibody has twelve CDRs altogether for binding antigens. In embodiments, the CAR molecules comprise one or more CDRs of MSLN, GPC-3, CD205, ALPP, and CD70

The present disclosure describes modified cells that include one or more different antigen binding domains. The modified cells can include at least two different antigen binding domains: a first antigen binding domain for expanding and/or maintaining the genetically modified cells, and a second antigen binding domain for killing a target cell, such as a tumor cell. For example, the first antigen binding domain binds a surface marker, such as a cell surface molecule of a white blood cell (WBC) (e.g., CD19), and the second antigen binding domain binds a target antigen on tumor cells. In embodiments, the cell surface molecule is a surface antigen of a WBC. In embodiments, the target antigen on tumor cells comprise one or more of MSLN, GPC-3, CD205, ALPP, and CD70. The at least two antigen binding domains may be located on the same or different modified cells. For example, the modified cells may include a modified cell including a CAR binding CD19, a modified cell including a CAR binding to ACPP, a modified cell including a CAR binding CD19 and ACPP, and/or a modified cell including two CARs that respectively bind CD19 and ACPP. In embodiments, the modified cells can be used to treat a subject having cancer.

In embodiments, the modified cells described herein includes a CAR molecule comprising at least two different antigen binding domains. The CAR molecule can be a bispecific CAR molecule. For example, the two antigen binding domains can be on the same CAR molecule, on different CAR molecules, or on a CAR molecule and T cell receptor (TCR). A single CAR can include at least two different antigen binding domains, or the two different antigen binding domains are each on a separate CAR molecule. The at least two different antigen binding domains can be on the same CAR molecule or different CAR molecules, but in the same modified cell. Moreover, the at least two different antigen binding domains can be on a CAR molecule and a T cell receptor in the same modified cell. In embodiments, the bispecific CAR molecule can include a binding domain binding an antigen of WBC (e.g., CD19) and a binding domain binding a solid tumor antigen. In embodiments, the bispecific CAR molecule may include two binding domains binding two different solid tumor antigens.

In embodiments, the at least two different antigen binding domains are on different CAR molecules which are expressed by different modified cells. Further, the one or more different antigen binding domains are on a CAR molecule and a T cell receptor, which are expressed by different modified cells.

Embodiments relate to compositions and methods of enhancing the activation ability and anti-tumor effect of CAR T cells. In embodiments, CD4 coreceptor may be incorporated into CAR T cells via the CAR molecule to enhance the activation ability of CAR T cells and to improve the clinical effect of CAR T cells. CD4 protein and CD8 (CD8A and CA8B dimers) have similar structures. For example, the extracellular domain of CD4 is composed of four globular IGG-like domains (D1-D4), which are responsible for recognizing its ligands (IL16, MHCII, HIV, and the like. The intracellular domain of CD4 is composed of 38 highly basic amino acids including palmitoylation sites and LCK binding sites. CD4 can stabilize the weak interaction between TCRs and MHC polypeptides, and recruit and deliver tyrosine kinase p56Lck to the TCR complex, which plays a role in the signal transmission and T cell activation process triggered by TCR. The intracellular domain of the CD8 dimer also contains LCK binding sites. Incorporation of CD4 or CD8 into a traditional CAR molecule enables the CAR to mediate the signal transduction and activation of the TCR complex by recruiting and binding LCK T cells. CD4 has a higher affinity when recombined with LCK, so that downstream genes are more effective. Further, the combination of CD4 and LCK can promote the redirection of thymocytes to MHC class II specific CD4 lineage T cells.

In the lymphatic system, CD8 can be expressed in two forms: a heterodimer formed by CD8α and CA8β or a homodimer formed by CD8α and CA8α. In embodiments, the co-receptor comprises the CD8αβ heterodimer. The end of the cytoplasmic domain of the CD8α chain contains the binding site for tyrosine kinase p56LCK, which is used to initiate the early TCR signal. The binding site is helpful for positioning the CD8 dimer in the lipid raft, as well as targeting, migrating, stabilizing, and functioning of the CD8 dimer. In embodiments, the intracellular domain of CD8 is incorporated into CAR T cells to enhance the activation ability of the CAR T cells. For example, a polynucleotide encoding the intracellular domain of CD8α and/or CD8β may be inserted into a CAR vector. In embodiments the intracellular domain of CD8α incorporated into CAR T cells may bind LCK through 9 amino acids containing the CxCP motif in the presence of zinc ions. In embodiments, the binding may be regulated by the activation of the CAR T cells. In embodiments, a polynucleotide encoding the above-mentioned 9 residues of the CD8α may be inserted into the CAR vector. The disclosure herein also describes designs of multiple repetitively connected sequences based on the above-mentioned 9 amino acids and incorporating them into CAR T cells for strengthening the CAR T cells' proliferation ability.

In embodiments, the intracellular domain of CD4 may be incorporated into CAR T cells to enhance the activation ability of the CAR T cells. For example, the intracellular domain of CD4 mainly binds to LCK through 17 amino acids residues including the CQCP motif, while the binding of LCK to other regions is optional. In embodiments, a polynucleotide encoding the 17 amino acid residues including the above-mentioned CQCP motif of CD4 may be introduced into the CAR vector to achieve the effect of enhancing the activation ability of CAR T cells. Also, embodiments include designs of multiple repetitively connected sequences based on the above-mentioned 17 amino acids and introduce the CAR vector into T cells, which further enhance the proliferation ability of CAR T cells.

Embodiments relate to an isolated nucleic acid sequence encoding a chimeric antigen receptor (CAR), wherein the CAR comprises an extracellular domain, a transmembrane domain, and an intracellular domain, wherein the intracellular domain comprises an intracellular domain of a co-receptor of a T cell receptor. Embodiments relate to a modified cell comprising a CAR and an exogenous polynucleotide encoding a peptide comprising an intracellular domain of a co-receptor of T cells and a transmembrane domain. Embodiments relate to an isolated nucleic acid sequence comprising a polynucleotide encoding the CAR and a polynucleotide encoding a peptide comprising an intracellular domain of a co-receptor of T cells and a transmembrane domain. Embodiments relate to a method of eliciting and/or enhancing T cell response in a subject having a solid tumor or treating a solid tumor in the subject, the method comprising administering an effective amount of T cells comprising the CAR. Embodiments relate to a vector comprising the isolated nucleic acid sequence. Embodiments relate to an isolated cell comprising the isolated nucleic acid sequence. Embodiments relate to a composition comprising a population of T cells comprising the CAR.

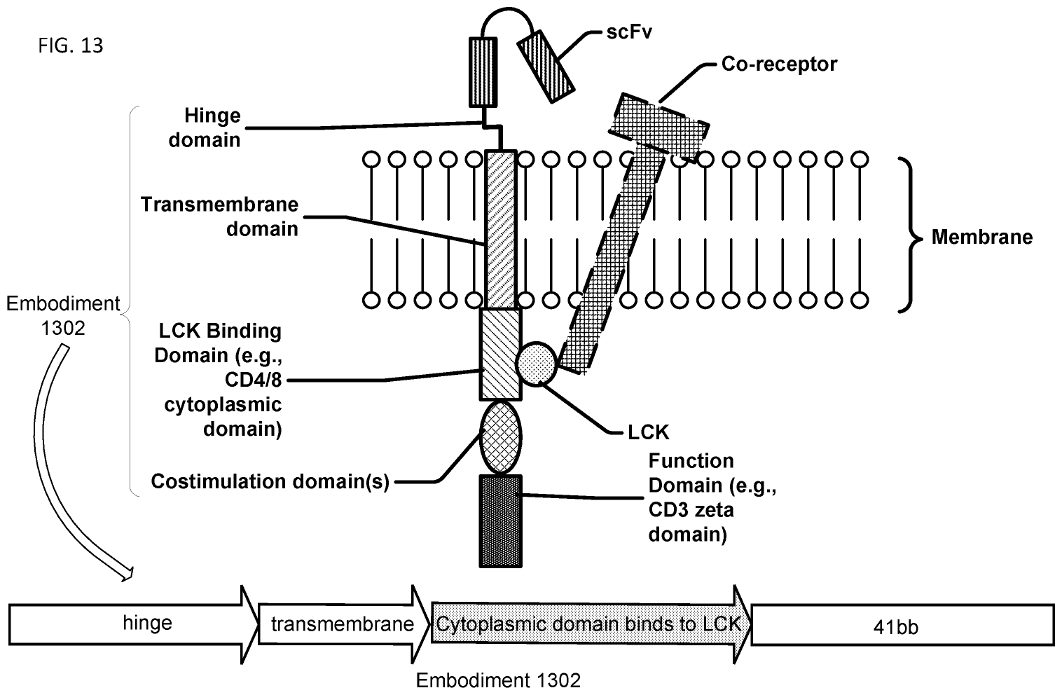

In embodiments, the CAR comprises a CD4 domain binding LCK. In embodiments, the CAR comprises the transmembrane domain GGxxG Motif in CD4. For example, the CD4 cytoplasm domain shown in FIG. 13 comprises the LCK binding site of the CD4 cytoplasm domain.

In embodiments, the co-receptor is CD4 or CD8. In embodiments, the extracellular domain binds an antigen of a tumor (e.g., solid tumor antigens listed in Table 1). In embodiments, the isolated nucleic acid sequence comprises one of the amino acid sequences of SEQ ID NOs: 1-5. In embodiments, the intracellular domain comprises a co-stimulatory signaling region comprising an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. In embodiments, the intracellular domain comprises a CD3 zeta signaling domain as the stimulatory signaling domain. In embodiments, the co-stimulatory signaling domain and the stimulatory signaling domain can be on different molecules in the same cell.

Interleukin-2 (IL-2) is the main cytokine used to culture cells for adoptive cell therapy, as it plays an important role in the proliferation and functional effect of T cells. For example, the CAR can be highly expressed in IL-2-cultured T cells three days after infection and expanded 100-fold in 2 weeks under IL-2 stimulation. While IL-2 is a central T cell cytokine that promotes T cell proliferation and effector function, toxicity due to its pluripotency limits its application to enhance CAR T cell immunotherapy (Zhang, Q., Hresko, M. E., Picton, L. K., et al. “A human orthogonal IL-2 and IL-2Rβ system enhances CAR T cell expansion and antitumor activity in a murine model of leukemia,” Science Translational Medicine, V. 13, No. 625, 2021.).

Embodiments of the present disclosure relate to composition and methods of enhancing activated CAR T cells' ability of releasing IL-2 by incorporating CD4 cytoplasm domains into CARs. Surprisingly, this enhanced ability is correlated with the manner of the incorporation of CD4 cytoplasm domains. For example, T cells including a CD4 cytoplasm domain inserted between a transmembrane domain and a co-stimulatory domain release more IL-2 than T cells with a CD4 cytoplasm domain inserted after a CD3 zeta domain.

Embodiments of the present disclosure relate to a polynucleotide encoding a CAR comprising a cytoplasmic domain of CD4, an intracellular domain of the CAR comprising SEQ ID NO: 17, wherein the cytoplasmic domain of CD4 is located between the transmembrane domain of the CAR and a stimulatory domain, such as CD3 zeta domain. In embodiments, the CAR comprises SEQ ID NO: 18 and a scFv targeting a solid tumor antigen.

In embodiments, the CAR comprises SEQ ID NO: 18 and a scFv comprising SEQ ID NO: 19 (GCC). In embodiments, the CAR comprises SEQ ID NO: 3.

Embodiments of the present disclosure relate to a method of enhancing IL-2 release by T cells expressing a CAR, the method comprising: obtaining a polynucleotide encoding a chimeric antigen receptor (CAR) comprising a cytoplasmic domain of CD4, and an intracellular domain of the CAR comprising SEQ ID NO: 17; introducing the polynucleotide into a T cell to obtain a CAR T cell; and contacting the CAR T cell with a cell or an antigen that the CAR binds, thereby causing release of IL-2, wherein an amount of IL-2 released is enhanced as compared to IL-2 released caused by a CAR T cell without a cytoplasmic domain of CD4 or CD8. In embodiments, in the CAR, the cytoplasmic domain of CD4 is located between the transmembrane domain and the stimulatory domain, such as the CD3 zeta domain. In embodiments, the CAR comprises SEQ ID NO: 18 and a scFv targeting a solid tumor antigen. In embodiments, the cytoplasmic domain of CD4 is replaced with the cytoplasmic domain of CD8.

CD4 and CD8, as a co-receptor of TCR, morphologically stabilizes the MHC-TCR complex by binding to MHC and recruits LCK signal to TCR to accelerate activation and promote the occurrence of cellular immunity. It seems that under weak activation conditions, the loss of LCK signaling can seriously affect CAR activation, especially 4-1BB CAR. The so-called weak activation conditions may be low levels of antigen-antibody binding or an inhibitory tumor environment such as negative regulatory mechanisms, insufficient energy supply, and the like. These situations are common in the tumor environment and may be related to the heterogeneity of the tumor and the complex negative regulatory environment.

Embodiments of the present disclosure relate to LCK binding CARs to resolve the problems mentioned above. Embodiments relates to a CAR comprising an antigen binding domain, a hinge domain, a transmembrane domain, and an intracellular domain that comprises a LCK binding domain. An example of the CAR is shown in FIG. 13. In embodiments, the LCK binding domain comprises at least a part of a cytoplasmic domain of the following AXL, CD2, CD4, CD5, CD8, CD8A (CD8α), CD8β (CD8β), CD44, CD45(PTPRC), or CD122(IL2RB). In embodiments, the LCK binding domain comprises at least a part of one of SEQ ID NO: 9, 14, and 29-34. In embodiments, the CAR comprises at least one of SEQ ID NOS: 1-5, 25-28, and 35-42.

In embodiments, the CAR comprises an extracellular domain, a transmembrane domain, and the intracellular domain. In embodiments, the intracellular domain comprises a co-stimulatory signaling region comprising an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. In embodiments, the CAR binds TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp 100, bcr-abl, tyrosinase, EphA2, Fucosyl GM1, sLe, GM3, TGS5, HMWMAA, o-acetyl-GD2, Folate receptor beta, T_{EM}1/CD248, T_{EM}7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In embodiments, the CAR T cell comprises a polynucleotide encoding one or more therapeutic agents comprising IL-1P, IL-2, IL-4, IL-5, IL-6, IL-8, IL-10, IL-12, IL-13, IL-15, IL-17, IL-1Ra, IL-2R, IFN-γ, IFN-γ, MIP-In, MIP-IP, MCP-1, TNFα, GM-CSF, CCL19, or MIP-Iα, GCSF, CXCL9, CXCL 10, CXCR factors, VEGF, RANTES, EOTAXIN, EGF, HGF, FGF-P, CD40, CD40L, or ferritin. In embodiments, the polynucleotide encoding the one or more therapeutic agents further encodes a VHL-interaction domain of HIF1α.

In embodiments, the CAR T cell comprises a polynucleotide encoding a dominant negative form of an inhibitory immune checkpoint molecule or a receptor thereof. In embodiments, the inhibitory immune checkpoint molecule is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), and CD 160. In embodiments, the inhibitory immune checkpoint molecule is modified PD-1. In embodiments, the modified PD-1 lacks a functional PD-1 intracellular domain for PD-1 signal transduction, interferes with a pathway between PD-1 of a human T cell and PD-L1 of a certain cell, comprises or is a PD-1 extracellular domain or a PD-1 transmembrane domain, comprises a modified PD-1 intracellular domain comprising a substitution or deletion as compared to a wild-type PD-1 intracellular domain, and/or comprises or is a soluble receptor comprising a PD-1 extracellular domain that binds to PD-L1 of a certain cell.

In embodiments, the CAR T cell comprises a polynucleotide encoding hTERT, SV40LT, or a combination thereof.

In embodiments, the CAR T cell has a reduced graft-versus-host disease (GVHD) response in a bioincompatible human recipient as compared to the GVHD response of a primary human T cell. In embodiments, the CAR T cell has a reduced expression of endogenous TRAC gene.

Embodiments of the present disclosure relate to a polynucleotide encoding a chimeric antigen receptor (CAR) comprising an extracellular domain, a transmembrane domain, and an intracellular domain comprising a cytoplasmic domain of CD4. In embodiments, the cytoplasmic domain comprises SEQ ID NO: 14 or 15. In embodiments, the intracellular domain further comprises a co-stimulatory signaling region in addition to the stimulatory signaling region, and the cytoplasmic domain of CD4 is between the transmembrane domain of the CAR and the co-stimulatory signaling region. In embodiments, the intracellular domain further comprises CD3 zeta domain as the stimulatory signaling region. In embodiments, the cytoplasmic domain of CD4 comprises a LCK binding site of the CD4. In embodiments, the intracellular domain of the CAR comprising SEQ ID NO: 17. In embodiments, the CAR comprises SEQ ID NO: 18 and a scFv targeting a solid tumor antigen.

Embodiments of the present disclosure relate to a polynucleotide encoding a chimeric antigen receptor (CAR) comprising an extracellular domain, a transmembrane domain, and the intracellular domain that comprises a cytoplasmic domain of CD8. In embodiments, the cytoplasmic domain comprises SEQ ID NO: 9, 10, or 11. In embodiments, the intracellular domain of the CAR comprising SEQ ID NO: 21, 22, or 23. In embodiments, the CAR comprises SEQ ID NO: 24 and a scFv targeting a solid tumor antigen.

Embodiments of the present disclosure relate to treating cancer using chimeric antigen receptor (CAR) cells. Embodiments relate to an isolated nucleic acid encoding a CAR, wherein the CAR comprises an extracellular domain, a transmembrane domain, and an intracellular domain, wherein the extracellular domain of the CAR binds an antigen of a solid tumor. For example, transcriptional data shows that expression of antigens such as SLC6A3, KISS1R, QRFPR in normal or healthy tissues is very low, but expression of such antigens in cells related to renal cancer is high.

The T cell response in a subject refers to cell-mediated immunity associated with a helper, killer, regulatory, and other types of T cells. For example, T cell response may include activities such as assistance to other white blood cells in immunologic processes and identifying and destroying virus-infected cells and tumor cells. T cell response in the subject may be measured via various indicators such as the number of virus-infected cells and/or tumor cells that T cells kill, an amount of cytokines that T cells release in co-culturing with virus-infected cells and/or tumor cells, a level of proliferation of T cells in the subject, a phenotype change of T cells (e.g., changes to memory T cells), and the longevity or lifespan of T cells in the subject.

In embodiments, in vitro killing assay may be performed by measuring the killing efficacy of CAR T cells by co-culturing CAR T cells with antigen-positive cells. CAR T cells may be considered to have killing effect on the corresponding antigen-positive cells by showing a decrease in the number of corresponding antigen-positive cells co-cultured with CAR T cells and an increase in the release of cytokines such as IFNγ, TNFα, and the like, as compared to control cells that do not express the corresponding antigen. Further, in vivo antitumor activity of the CAR T cells may be tested. For example, xenograft models can be established using the antigens described herein in immunodeficient mice.

Heterotransplantation of human cancer cells or tumor biopsies into immunodeficient rodents (xenograft models) has, for the past two decades, constituted the major preclinical screen for the development of novel cancer therapeutics (Song et al., Cancer Res. PMC 2014 Aug. 21, and Morton et al., Nature Protocols, 2, -247-250 (2007)). To evaluate the anti-tumor activity of CAR T cells in vivo, immunodeficient mice bearing tumor xenografts were evaluated for CAR T cell anti-tumor activity, for example, a decrease in mouse tumors and/or mouse blood cytokines, such as IFNγ, TNFα, and the like.

Embodiments of this present disclosure relate to a method of inhibiting tumor growth and/or treating a subject with a cancer form. The method comprises administering a modified immune cell that binds a tumor antigen associated with a form of cancer and one or more therapeutic agents to the subject, wherein the one or more therapeutic agents are listed in Table 2, and the tumor antigen and the corresponding form of cancer are listed in Table 1.

Embodiments of this present disclosure relate to a method of enhancing anti-tumor activity and/or treatment of a form of cancer. The method comprises contacting a modified immune cell that binds a tumor antigen associated with a form of cancer and one or more therapeutic agents with a cell expressing the tumor antigen, wherein the one or more therapeutic agents are listed in Table 2 and the tumor antigen and the corresponding form of cancer is listed in Table 1, and wherein the anti-tumor activity and/or the treatment of the form of cancer is enhanced as compared to contacting the cell expressing the tumor antigen with the modified immune cell or the therapeutic agent, separately.

Embodiments of this present disclosure relate to a kit for treating a tumor. The kit comprises modified immune cells that binds a tumor antigen associated with a form of cancer and one or more therapeutic agents for administering to a subject in need thereof, wherein the one or more therapeutic agents are listed in Table 2 and the tumor antigen and corresponding form of cancer is listed in Table 1.

In embodiments, the modified immune cell is a modified NK, DC, macrophage, or T cell. In embodiments, the modified immune cell comprises a CAR or a modified TCR. In embodiments, the method comprises administering one or more therapeutic agents first and then administering the modified immune cell, simultaneously administering the modified immune cell and the one or more therapeutic agents, and administering the modified immune cell and then administering the one or more therapeutic agents.

TABLE 2

| Therapeutic agents | | Cancer forms |
|---|---|---|
| CXC chemokine receptor 4 (CXCR4) blockade (BL-8040), | | metastatic |
| pembrolizumab(PD1), NAPOLI-1 chemotherapy regimen (liposomal | | pancreatic ductal |
| irinotecan, fluorouracil and leucovorin) | | adenocarcinoma |
| tigatuzumab(humanized monoclonal antibody TRA-8 that binds to | | unresectable or |
| the death receptor 5 and induces apoptosis of human cancer cell | | metastatic |
| lines via the caspase cascade) + gemcitabine | | pancreatic cancer |
| agonistic anti-CD40 mAb (CP-870,893) + gemcitabine | | advanced |
| | | pancreatic ductal |
| | | adenocarcinoma |
| | | (PDA) |
| agonist CD40 antibody(CP-870,893) + gemcitabine | | advanced |
| | | pancreatic ductal |
| | | adenocarcinoma |
| CD40 agonistic monoclonal antibody APX005M together with | | untreated |
| gemcitabine (Gem) and nab-paclitaxel (NP) with or without | | metastatic ductal |
| nivolumab (PD1)(Nivo) | | pancreatic |
| | | adenocarcinoma |
| | | (PDAC) |
| CD40 agonistic monoclonal antibody APX005M (sotigalimab) and | | metastatic |
| chemotherapy(gemcitabine plus nab-paclitaxel), with or without | | pancreatic |
| nivolumab | | adenocarcinoma |
| Stereotactic body radiotherapy plus pembrolizumab(PD1) and | | postoperative |
| trametinib versus stereotactic body radiotherapy plus gemcitabine | | locally recurrent |
| | | pancreatic cancer |
| TGFβ receptor I kinase inhibitor galunisertib plus the anti-PD-L1 | | metastatic |
| antibody durvalumab | | pancreatic cancer |
| gemcitabine, trastuzumab(Her2) and erlotinib as first-line treatment | | metastatic |
| | | pancreatic |
| | | adenocarcinoma |
| trastuzumab(Her2) and capecitabine | | metastatic |
| | | pancreatic cancer |
| | | and HER2 |
| | | overexpression |
| Andecaliximab (GS-5745), a monoclonal antibody targeting | | advanced |
| MMP9 + gemcitabine + nab-paclitaxel | | pancreatic |
| | | adenocarcinoma |
| Gemcitabine/nab-paclitaxel with pamrevlumab(anti-CTGF) | | locally advanced |
| | | pancreatic cancer |
| nab-paclitaxel and gemcitabine with tarextumab (a fully human | | untreated |
| IgG2 antibody that inhibits Notch2/3 receptors) | | metastatic |
| | | pancreatic cancer |
| Wnt Inhibitor Ipafricept with Gemcitabine and nab-paclitaxel | | untreated |
| | | metastatic |
| | | pancreatic |
| | | adenocarcinoma |
| | | (mPDAC) |
| Wnt pathway inhibitor vantictumab in combination with nab-paclitaxel | | previously |
| and gemcitabine | | untreated |
| | | metastatic |
| | | pancreatic cancer |
| granulocyte-macrophage colony-stimulating factor (GM-CSF)- | | metastatic |
| allogeneic pancreatic tumor cells (GVAX) and ipilimumab(CTLA-4) | | pancreatic ductal |
| | | adenocarcinoma |
| GVAX pancreas vaccine (GM-CSF-secreting allogeneic pancreatic | | metastatic |
| tumor cells) with cyclophosphamide (Cy) and CRS-207 (live, | | pancreatic cancer |
| attenuated Listeria monocytogenes-expressing mesothelin). In the | | |
| current study, we compared Cy/GVAX followed by CRS-207 with | | |
| (Arm A) or without nivolumab (Arm B). | | |
| nivolumab (PD1) plus nab-paclitaxel and gemcitabine | | Advanced |
| | | Pancreatic |
| | | Cancer |
| gemcitabine, nab-paclitaxel, and pembrolizumab(PD1) | | metastatic |
| | | pancreatic |
| | | adenocarcinoma |
| Bruton tyrosine kinase inhibitor acalabrutinib, alone or with | | advanced |
| pembrolizumab (PD1) | | pancreatic cancer |
| Pembrolizumab(PD1) in Combination with the Oncolytic Virus | | Advanced |
| Pelareorep and Chemotherapy (5-fluorouracil, gemcitabine, or | | Pancreatic |
| irinotecan) | | Adenocarcinoma |
| Ipilimumab(CTLA4) and Gemcitabine | | Advanced |
| | | Pancreatic |
| | | Cancer |
| tremelimumab (CTLA4), gemcitabine | | |
| Mesothelin-targeted Immunotoxin LMB-100 with Nab-Paclitaxel | | Advanced |
| | | Pancreatic |
| | | Adenocarcinoma |
| Istiratumab(MM-141) a fully human tetravalent bispecific antibody | | metastatic |
| (IGF-1R + ErbB3), plus nab-paclitaxel and gemcitabine | | pancreatic cancer |
| gemcitabine/leucovorin/fluorouracil/oregovomab (anti-CA-125) | | Locally Advanced |
| | | Pancreatic |
| | | Adenocarcinoma |
| gemcitabine + dasatinib (gd) or gemcitabine + dasatinib + cetuximab | | pancreatic |
| (EGFR) (GDC) | | adenocarcinoma |
| Panitumumab (EGFR), Erlotinib, and Gemcitabine Versus Erlotinib | | Untreated, |
| and Gemcitabine | | Metastatic |
| | | Pancreatic |
| | | Adenocarcinoma |
| nimotuzumab (EGFR), Gemcitabine | | KRAS wildtype |
| | | patients with |
| | | locally advanced |
| | | or metastatic |
| | | pancreatic cancer |
| capecitabine, erlotinib and bevacizumab (VEGF) | | pancreatic |
| | | adenocarcinoma |
| Bevacizumab, Erlotinib and Capecitabine | | Unresectable |
| | | Pancreatic |
| | | Cancer |
| gemcitabine plus IGF-1R antagonist (MK-0646) versus gemcitabine | | advanced |
| plus erlotinib with and without MK-0646 | | pancreatic |
| | | adenocarcinoma |
| Ganitumab (insulin-like growth factor Type 1 receptor), gemcitabine | | as first-line |
| | | therapy in |
| | | patients with |
| | | metastatic |
| | | pancreatic cancer |
| Simtuzumab(LOXL2) or Placebo in Combination with Gemcitabine | | Pancreatic |
| for the First-Line Treatment | | Adenocarcinoma |
| gemcitabine, cisplatin, and fluorouracil plus bevacizumab(VEGF) | | locally advanced |
| and cetuximab(EGFR) | | or metastatic |
| | | pancreatic cancer |
| Irinotecan/Docetaxel or Irinotecan/Docetaxel Plus Cetuximab(EGFR) | | Metastatic |
| | | Pancreatic |
| | | Cancer |
| LY2495655 (antimyostatin antibody) | | stage II-IV |
| plus standard-of-care chemotherapy | | pancreatic cancer |
| liposomal irinotecan + 5 fluorouracil/leucovorin (5 FU/LV) | | metastatic |
| | | pancreatic ductal |
| | | adenocarcinoma |
| VCN-01 (an oncolytic adenovirus designed to replicate in cancer cells | | pancreatic |
| with a dysfunctional RB1 pathway and express hyaluronidase) plus | | cancer. |
| chemotherapy (either gemcitabine or nab-paclitaxel plus | | |
| gemcitabine) | | |
| Arsenic trioxide | | Pancreatic cancer |
| Retinoic acid | | |
| olapani | | breast cancer |
| Rucapani | | |
| Nirapani | | |
| Talazopanib | | |
| NKTR-214 + Nivolumab | | Various |
| N-803 + BCG | | Various |
| MPLA + IFN-g | | Various |
| lenalidomide | | Various |
| Bevacizumab | | Various |
| Ramucirumab | | |
| ranibizumab | | |
| Aflibercept | | |
| Compaq | | |
| Paclitaxel | | Various |
| gemcitabine etc. | | |
| Keytruda | | Various |
| Yeryoy | | |
| Tecentriq | | |
| Imfinzi | | |
| Sorafenib | | Various |
| ilixadencel | | Kidney cancer, |
| | | liver cancer, |
| | | gastrointestinal |
| | | stromal tumors, |
| | | head and neck |
| | | tumors, non-small |
| | | cell lung cancer |
| | | and gastric |
| | | cancer |
| zydelig | | Various |
| aliqopa | | |
| copiktra | | |
| Apelis | | |
| Decitabine | | Various |
| lenalidomide | | Various |
| Pembrolizumab | Pemetrexed Pemetrexed + platinum platinum | EGFR mutation- |
| (K drug) | | negative or ALK - |
| | | negative |
| | | metastatic non- |
| | | squamous |
| | | NSCLC |
| | Carboplatin carboplatin + paclitaxel/albumin | metastatic |
| | paclitaxel/nab-paclitaxel | squamous |
| | | NSCLC |
| | Lenvatinib_{—} | Advanced Renal |
| | | Cell Carcinoma |
| | | (RCC) |
| | | Unresectable |
| | | hepatocellular |
| | | carcinoma |
| | | (HCC) |
| | | advanced |
| | | endometrial |
| | | cancer |
| | nab- paclitaxel; paclitaxel; or gemcitabine plus | triple negative |
| | carboplatin | breast cancer |
| | Axitinib INLYTA_{—} | Advanced renal |
| | | cell carcinoma |
| | | RCC |
| | Cisplatin and Fluorouracil | Advanced/ |
| | | unresectable or |
| | | metastatic |
| | | adenocarcinoma |
| | | or esophageal |
| | | squamous cell |
| | | carcinoma |
| | | (ESCC) or |
| | | Siewert type 1 |
| | | esophagogastric |
| | | junction |
| | | adenocarcinoma |
| | | (EGJ) |
| Avelumab | Axitinib INLYTA_{—} | Advanced renal |
| (B drug) | | cell carcinoma |
| | | RCC |
| Durvalumab | Etoposide + carboplatin or cisplatin | Extensive stage |
| (I drug) | | small cell lung |
| | | cancer (ES- |
| | | SCLC) |
| Atezolizumab | Carboplatin + Etoposide | Extensive stage |
| (T drug) | | small cell lung |
| | | cancer (ES- |
| | | SCLC) |
| | cobimetinib and vemurafenib_{——} | BRAF V600 |
| | | mutation-positive |
| | | advanced |
| | | melanoma |
| | Avastin (bevacizumab) and chemotherapy | squamous non- |
| | (paclitaxel + carboplatin) | small cell lung |
| | | cancer (NSq |
| | | NSCLC) without |
| | | EGFR or ALK |
| | | mutations |
| | Albumin Paclitaxel | PD-L1- positive |
| | | unresectable |
| | | locally advanced |
| | | or metastatic |
| | | triple-negative |
| | | breast cancer |
| | | (TNBC) |
| Camrelizumab_{———} | Cisplatin and gemcitabine | Locally recurrent |
| | | or metastatic |
| | | nasopharyngeal |
| | | carcinoma |
| | Taxone and Cisplatin | Unresectable |
| | | locally advanced/ |
| | | recurrent or |
| | | metastatic |
| | | esophageal |
| | | squamous cell |
| | | carcinoma |
| | Taxone and carboplatin | locally advanced |
| | | or metastatic |
| | | squamous non- |
| | | small cell lung |
| | | cancer |
| | Pemetrexed and carboplatin | Epidermal growth |
| | | factor receptor |
| | | (EGFR) gene |
| | | mutation-negative |
| | | and anaplastic |
| | | lymphoma kinase |
| | | (ALK)-negative, |
| | | unresectable |
| | | locally advanced |
| | | or metastatic non- |
| | | squamous non- |
| | | small cell lung |
| | | cancer (NSCLC) |
| Pembrolizumab | Lenvatinib | melanoma |
| (K drug) | | urothelial |
| | | carcinoma |
| | | head and neck |
| | | squamous cell |
| | | carcinoma |
| Camrelizumab_{———} | Combination of apatinib and chemotherapy | Esophageal |
| | (paclitaxel liposome + nedaplatin) | squamous cell |
| | | carcinoma |
| | famitinib and nab-paclitaxel | Advanced |
| | | immunomodulatory |
| | | triple-negative |
| | | breast cancer |
| Atezolizumab | Bevacizumab + pemetrexed and carboplatin | EGFR - mutant |
| (T drug) | | NSCLC after TKI |
| | | failure |
| Durvalumab | tremelimumab + platinum-based | Non-Small Cell |
| (I drug) | chemotherapy | Lung Cancer |
| | | NSCLC |
| Nivoliumab | Capecitabine and oxaliplatin every 3 weeks or | advanced or |
| (O drug) | folinic acid, fluorouracil and oxaliplatin every 2 | metastatic |
| | weeks | gastric, GEJ and |
| | | esophageal |
| | | adenocarcinoma |
| | Lenvatinib | Unresectable |
| | | hepatocellular |
| | | carcinoma |
| | | (HCC) |
| PDR001 | Tafinlar + Mekinist | BRAF V600- |
| (spartalizumab) | | Mutant |
| | | Unresectable or |
| | | Metastatic |
| | | Melanoma |
| Avelumab | Platinum + Paclitaxel | ovarian cancer |
| (B drug) | PEGylated Doxorubicin Liposomes (PLD) | Ovarian cancer |
| | | resistant or |
| | | refractory to |
| | | platinum |
| | | chemotherapy |
| | Docetaxel_{—} | lung cancer |
| Pembrolizumab | Gemcitabine | advanced |
| (K drug) | | urothelial |
| | | carcinoma |
| Durvalumab | Osimertinib | NSCLC |
| (I drug) | | |
| Atezolizumab | Carboplatin + paclitaxel or nab-paclitaxel | Advanced |
| (T drug) | | squamous |
| | | NSCLC |
| | paclitaxel | PD-L1- positive |
| | | metastatic triple- |
| | | negative breast |
| | | cancer (TNBC) |
| Nivolumab | VB-111 | CRC |
| Bevacizumab | 5-FU | CRC |
| Bevacizumab/ | FOLFOX, 5-FU | CRC |
| Atezolizumab | | |
| Bevacizumab | Trifluridine/tipiracil | CRC |
| Bevacizumab | FOLFOX | CRC |
| Bevacizumab/ | FOLFOX | CRC |
| Atezolizumab | | |
| Bevacizumab + | Capecitabine | CRC |
| Atezolizumab | | |
| Bevacizumab | Capecitabine | CRC |
| Cetuximab + | FOLFOX | CRC |
| Avelumab | | |
| Nivolumab + | Temozolomide | CRC |
| Ipilimumab + | | |
| Pembrolizumab | | |
| Nivolumab | SOC(Standard Of Care) chemo | Lung Cancer |
| Nivolumab + | SOC(Standard Of Care) chemo | Lung Cancer |
| Ipilimumab | | |
| Nivolumab + | SOC(Standard Of Care) chemo: carboplatin + | Lung Cancer |
| Ipilimumab | paclitaxel or carboplatin + pemetrexed or | |
| | cisplatin + pemetrexed | |
| pembrolizumab | carboplatin + pemetrexed | Lung Cancer |
| pembrolizumab | platinum derivative + pemetrexed | Lung Cancer |
| pembrolizumab | carboplatin + paclitaxel/nab paclitaxel | Lung Cancer |
| Atezolizumab | carboplatin + nab paclitaxel | Lung Cancer |
| Atezolizumab | platinum derivative + pemetrexed | Lung Cancer |
| Atezolizumab + | carboplatin + paclitaxel | Lung Cancer |
| Bevacizumab | | |

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples, but rather, should be construed to encompass any and all variations which become evident as a result of the teaching provided herein.

### Exemplary Embodiments

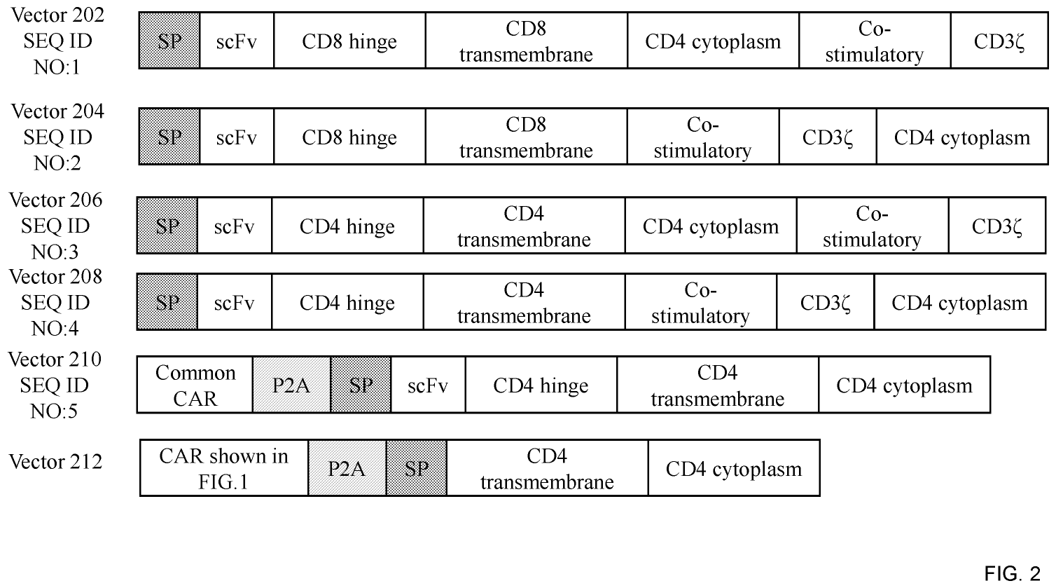

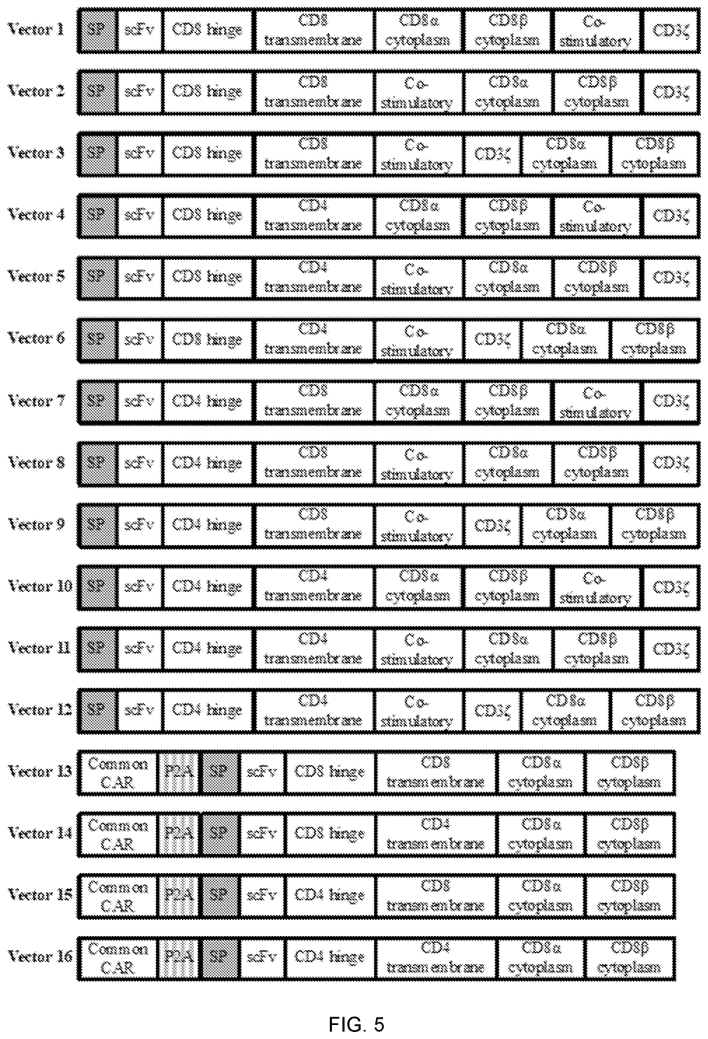

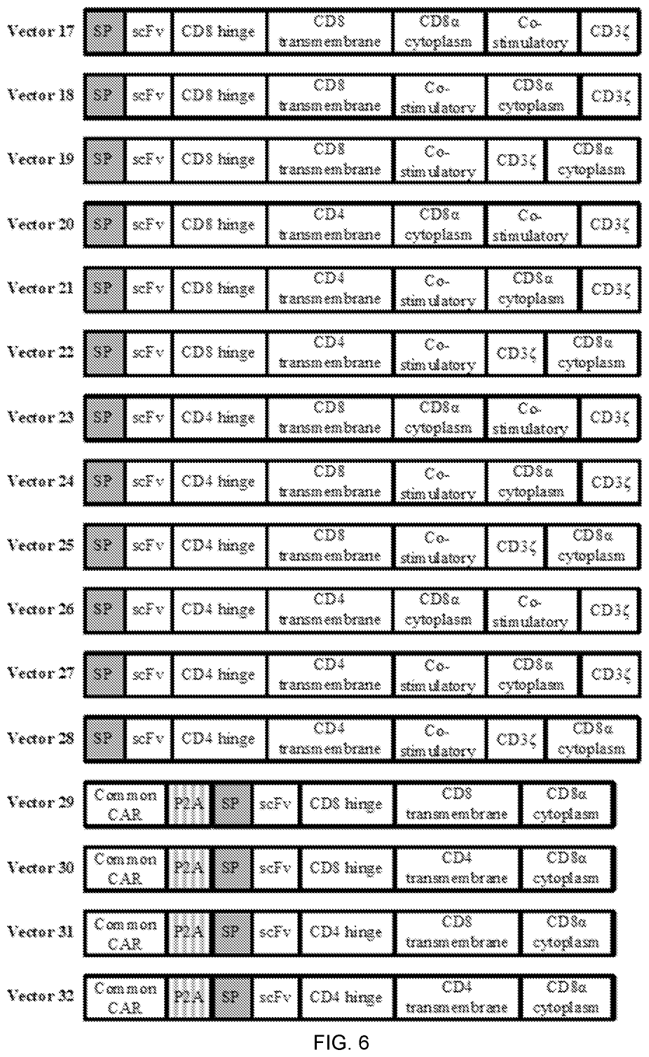

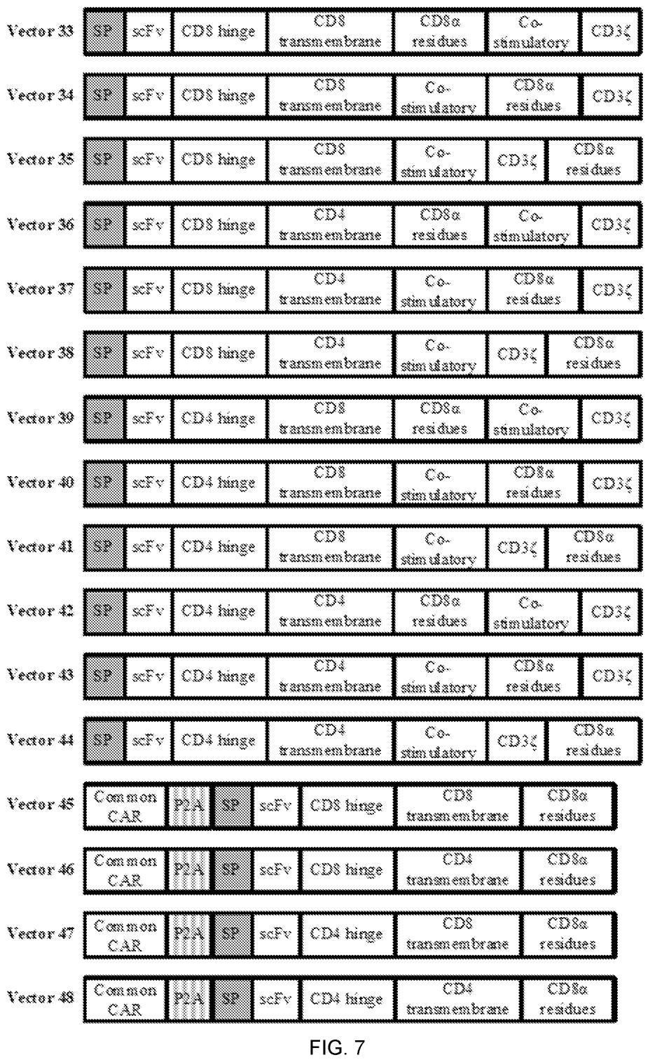

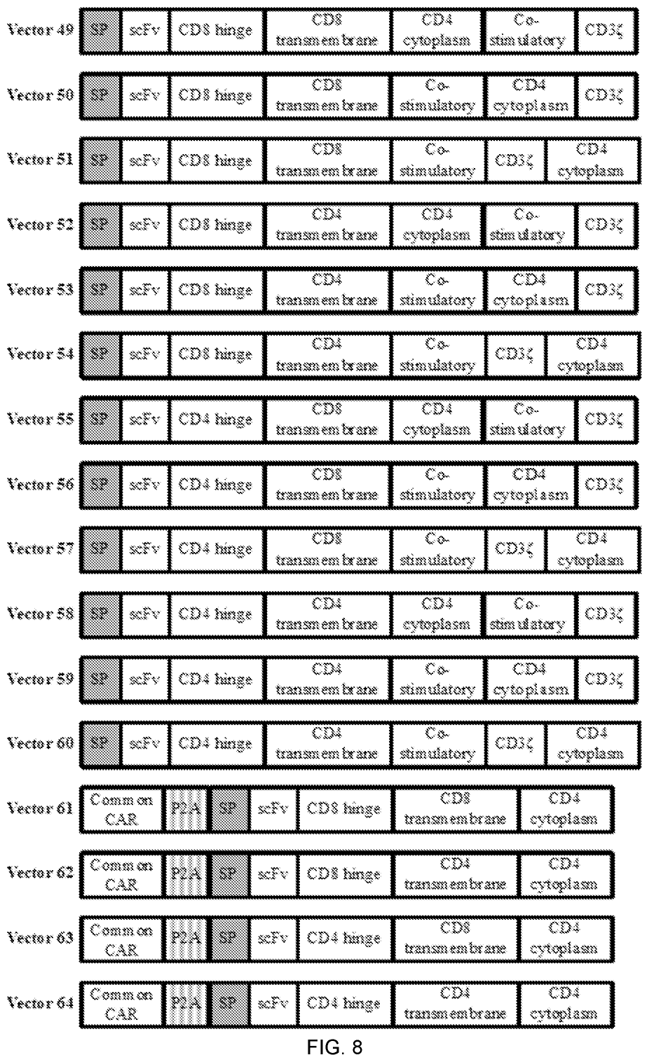

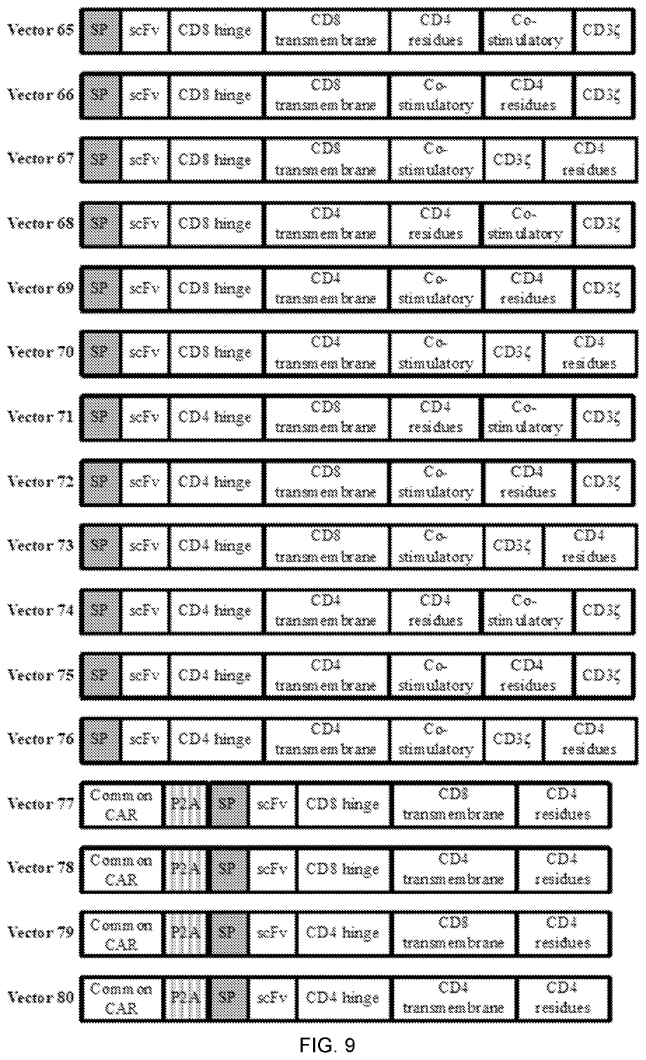

The following are exemplary embodiments:
1. An isolated nucleic acid encoding a chimeric antigen receptor (CAR), wherein the CAR comprises an extracellular domain, a transmembrane domain, and an intracellular domain, wherein the intracellular domain comprises a stimulatory signaling domain and an intracellular domain of a co-receptor of a T cell receptor, for example, as shown in FIGS. 2 and 5-9.
2. The isolated nucleic acid of embodiment 1, wherein the co-receptor is CD4 or CD8.
3. The isolated nucleic acid of embodiment 1 or 2, wherein the extracellular domain binds an antigen of a tumor, for example, one of the solid tumor antigens listed in Table 1.
4. The isolated nucleic acid of any of embodiments 1-3, wherein the isolated nucleic acid comprises one of the amino acid sequences of SEQ ID NOs: 1-5.
5. A method of eliciting and/or enhancing T cell response or NK cell response in a subject having a solid tumor or treating a solid tumor in the subject, the method comprising administering an effective amount of modified cells (e.g., T cells and/or NK cells) comprising a CAR encoded by the nucleic acid of any one of embodiments 1-4.
6. The isolated nucleic acid or the method of any one of embodiments 1-5, wherein the intracellular domain comprises a co-stimulatory signaling region comprising an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.
7. The isolated nucleic acid or the method of any one of embodiments 1-6, wherein the intracellular domain comprises a CD3 zeta signaling domain.
8. A vector comprising the isolated nucleic acid of any one of embodiments 1-7.
9. An isolated cell comprising the isolated nucleic acid of any one of embodiments 1-8.
10. A composition comprising a population of T cells comprising the CAR encoded by the nucleic acid or vector of any one of embodiments 1-4 or 6-9.
11. A CAR encoded by the isolated nucleic acid or vector of any one of embodiments 1˜4 or 6-9.
12. An isolated nucleic acid encoding a chimeric antigen receptor (CAR), wherein the CAR comprises an extracellular domain, a transmembrane domain, and an intracellular domain, wherein the extracellular domain binds a gene fusion antigen, for example a neoantigen.
13. The isolated nucleic acid, the vector, the composition, the CAR, or the method of any one of embodiments 1-12, wherein the intracellular domain comprises a co-stimulatory signaling region comprising an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.
14. The isolated nucleic acid, the vector, the composition, the CAR, or the method of any one of embodiments 1-13, wherein the intracellular domain comprises a CD3 zeta signaling domain.
15. An isolated cell comprising the nucleic acid, the vector, or the CAR of any a suitable embodiment of any one of embodiments 1-15, or a composition comprising the isolated nucleic acid, the vector, or the CAR of any one of suitable embodiments 1-14.
16. A modified cell comprising a CAR and an exogenous polynucleotide encoding a peptide or a fusion protein comprising an intracellular domain of a co-receptor of T cells and a transmembrane domain.
17. An isolated nucleic acid encoding a CAR and encoding a peptide comprising an intracellular domain of a co-receptor of T cells and a transmembrane domain.
18. The modified cell or the isolated nucleic acid of embodiment 16 or 17, wherein the co-receptor is CD4 or CD8.
19. The isolated nucleic acid, the vector, the CAR, the modified T cell, the composition, or the method of any one of embodiments 1-18, wherein the cell or modified cell is a T cell derived from a healthy donor or a subject having cancer, and the modified T cell comprises a dominant negative form of a receptor associated with an immune checkpoint inhibitor.
20. The isolated nucleic acid, the vector, the CAR, the modified T cell, the composition, or the method of embodiment 19, wherein the immune checkpoint inhibitor is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), and CD 160.
21. The isolated nucleic acid, the vector, the CAR, the modified T cell, the composition, or the method of embodiment 19 or 20, wherein the immune checkpoint inhibitor is modified PD-1.
22. The isolated nucleic acid, the vector, the CAR, the modified T cell, the composition, or the method of any one of embodiments 19-21, wherein the modified PD-1 lacks a functional PD-1 intracellular domain for PD-1 signal transduction, interferes with a pathway of PD-1 of a human T cell and PD-L1 of a certain cell, comprises or is a PD-1 extracellular domain, comprises a PD-1 transmembrane domain, comprises a modified PD-1 intracellular domain comprising a substitution or deletion as compared to a wild-type PD-1 intracellular domain, comprises or is a soluble receptor comprising a PD-1 extracellular domain that binds PD-L1 of a certain cell, or a combination thereof.
23. The isolated nucleic acid, the vector, the CAR, the modified T cell, the composition, or the method of any one of embodiments 19-22, wherein an inhibitory effect of PD-L1 on cytokine production of the human T cells of the population is less than an inhibitory effect of PD-L1 on cytokine production of human T cells that do not comprise at least a part of the nucleic acid encoding the modified PD-1.
24. The isolated nucleic acid, the vector, the CAR, the modified T cell, the composition, or the method of any one of embodiments 1-23, wherein the modified T cell is engineered to express and secrete a therapeutic agent such as a cytokine.
25. The isolated nucleic acid, the vector, the CAR, the modified T cell, the composition, or the method of embodiment 24, wherein the therapeutic agent that is or comprises IFN-γ.
26. The isolated nucleic acid, the vector, the CAR, the modified T cell, the composition, or the method of embodiment 24, wherein the therapeutic agent is or comprises at least one of IL-6, IFN-γ, IL-17, and CCL19.
27 The isolated nucleic acid, the vector, the CAR, the modified T cell, the composition, or the method of embodiment 24, wherein the therapeutic agent is or comprises IL-15 or IL-12, or a combination thereof.
28. The isolated nucleic acid, the vector, the CAR, the modified T cell, the composition, or the method of embodiment 24, wherein the therapeutic agent is or comprises a recombinant or native cytokine.
29. The isolated nucleic acid, the vector, the CAR, the modified T cell, the composition, or the method of embodiment 24, wherein the therapeutic agent comprises a FC fusion protein associated with a small protein.
30. The isolated nucleic acid, the vector, the CAR, the modified T cell, the composition, or the method of embodiment 24, wherein the small protein is or comprises IL-12, IL-15, IL-6 or IFN-γ.
31. The isolated nucleic acid, the vector, the CAR, the modified T cell, the composition, or the method of embodiment 24, wherein the therapeutic agent is regulated by Hif1a, NFAT, FOXP3, and/or NFkB.
32. The isolated nucleic acid, the vector, the CAR, the modified T cell, the composition, or the method of any one of embodiments 24-32, wherein the therapeutic agent is or comprises two or more recombinant or native cytokines that are connected via 2A and/or IRES component.
33. The isolated nucleic acid, the vector, the CAR, the modified T cell, the composition, or the method of any one of embodiments 1-32, wherein the modified T cell comprises a first targeting vector and a second targeting vector, the first targeting vector comprising a nucleic acid encoding a CAR binding a blood antigen and the therapeutic agent, and the second targeting vector comprises a nucleic acid encoding a CAR binding a solid tumor antigen and a dominant negative form of an immune checkpoint molecule.
34. The isolated nucleic acid, the vector, the CAR, the modified T cell, the composition, or the method of any one of embodiments 1-33, wherein the modified T cell comprises a first targeting vector and a second targeting vector, the first targeting vector comprising a nucleic acid encoding a CAR binding CD19 and the therapeutic agent, and the second targeting vector comprises a nucleic acid encoding a CAR binding UPK2, ACPP, SIGLEC15, or KISS1R and a dominant negative form of PD-1.
35. The isolated nucleic acid, the vector, the CAR, the modified T cell, the composition, or the method of any one of embodiments 1-32, wherein the modified T cell comprises a first targeting vector and a second targeting vector, the first targeting vector comprising a nucleic acid encoding a CAR binding a blood antigen, and the second targeting vector comprises a nucleic acid encoding a CAR a binding solid tumor antigen.
36. The isolated nucleic acid, the vector, the CAR, the modified T cell, the composition, or the method of one of embodiments 1-32, wherein the modified T cell comprises a first targeting vector and a second targeting vector, the first targeting vector comprising a nucleic acid encoding a CAR binding a B cell antigen, and the second targeting vector comprises a nucleic acid encoding a CAR binding a solid tumor antigen.
37. The isolated nucleic acid, the vector, the CAR, the modified T cell, the composition, or the method of one of embodiments 1-36, wherein the modified T cell comprises a nucleic acid encoding hTERT, SV40LT, or a combination thereof.
38. The isolated nucleic acid, the vector, the CAR, the modified T cell, the composition, or the method of embodiment 37, wherein the modified T cell is more proliferable than T cells without the nucleic acid encoding hTERT, SV40LT, or a combination thereof.
39. The isolated nucleic acid, the vector, the CAR, the modified T cell, the composition, or the method of embodiment 38, wherein the proliferable cell retains functions of normal T cells/CAR T cells such as cell therapy functions.
40. The isolated nucleic acid, the vector, the CAR, the modified T cell, the composition, or the method of any one of embodiments 37-39, wherein the T cell comprises a CAR and is cultured in the presence of an agent that is recognized by the extracellular domain of the CAR, thereby producing a modified CAR cell.
41. The isolated nucleic acid, the vector, the CAR, the modified T cell, the composition, or the method of any one of embodiments 1-40, wherein nucleic acid encoding hTERT, nucleic acid encoding SV40LT, or a combination thereof is genomically integrated and wherein there is constitutive expression of hTERT, SV40LT, or a combination thereof.
42. The isolated nucleic acid, the vector, the CAR, the modified T cell, the composition, or the method of any one of embodiments 1-41, wherein expression of hTERT, SV40LT, or a combination thereof, is regulated by an inducible expression system such as a rtTA-TRE system.
43. The isolated nucleic acid, the vector, the CAR, the modified T cell, the composition, or the method of any one of embodiments 1-42, wherein modified T cell comprises a nucleic acid encoding a suicide gene such as a an HSV-TK system.
44. The isolated nucleic acid, the vector, the CAR, the modified T cell, the composition, or the method of any one of embodiments 1-43, wherein the cell has a reduced graft-versus-host disease (GVHD) response in a bioincompatible human recipient as compared to the GVHD response of the primary human T cell.
45. The isolated nucleic acid, the vector, the CAR, the modified T cell, the composition, or the method of one of embodiments 1-44, wherein the cell has a reduced expression of endogenous TRAC gene.
46. The isolated nucleic acid, the vector, the CAR, the modified T cell, the composition, or the method of one of embodiments 1-45, wherein the CAR comprises a scFv or CAR sequence listed in Table 2.
47. A population of cells comprising the one or more modified cells of any one of the suitable embodiments of embodiments 1-46.
48. Use of the nucleic acids, the CAR, the antibodies, the vectors, the cells, the modified T cells, the population of cells, the compositions, the pharmaceutical compositions, the kit, or the methods of any one of the suitable embodiments of embodiments 1-47 or described herein in this disclosure for use in a method of treating a subject in need thereof.
49. The use of embodiment 48, wherein the subject is a human or animal.
50. The use of embodiment 48 or 49, wherein the subject is suffering from cancer.
51. The use of any one of embodiments 48-50, wherein the use elicits and/or enhances a T cell response in the subject.
52. Use of the nucleic acids, the CAR molecules, the antibodies, the vectors, the cells, the population of cells, the compositions, the pharmaceutical compositions, the kit, or the methods of any one of the suitable embodiments of embodiments 1-51 or described herein for use in a method of eliciting and/or enhancing a T cell response in a subject.
53. The use of embodiment 52, wherein the subject is a human or animal.
54. The use of embodiment 52 or 53, wherein the subject is suffering from cancer.
55. The isolated nucleic acid, the vector, the CAR, the modified T cell, the composition, the method, or the use of any one of embodiments 1-53, wherein the CD4 cytoplasmic domain is between the transmembrane domain of the CAR and the CD3 zeta domain.
56. The method of any suitable preceding embodiments, wherein the T cell response comprises T cell activation.
57. The method of embodiment 56, wherein a level of the T cell activation is measured based on at least one of: expression of CD40L on T cells, an amount of IL-2 released, and expression of Glucose transporter 1 (Glut1).
58. The method of embodiment 56 or 57, wherein the CD4 cytoplasmic domain is located between the transmembrane domain of the CAR and the CD3 zeta domain.
59. A method of enhancing IL-2 release by lymphocytes expressing a CAR in vivo, the method comprising:

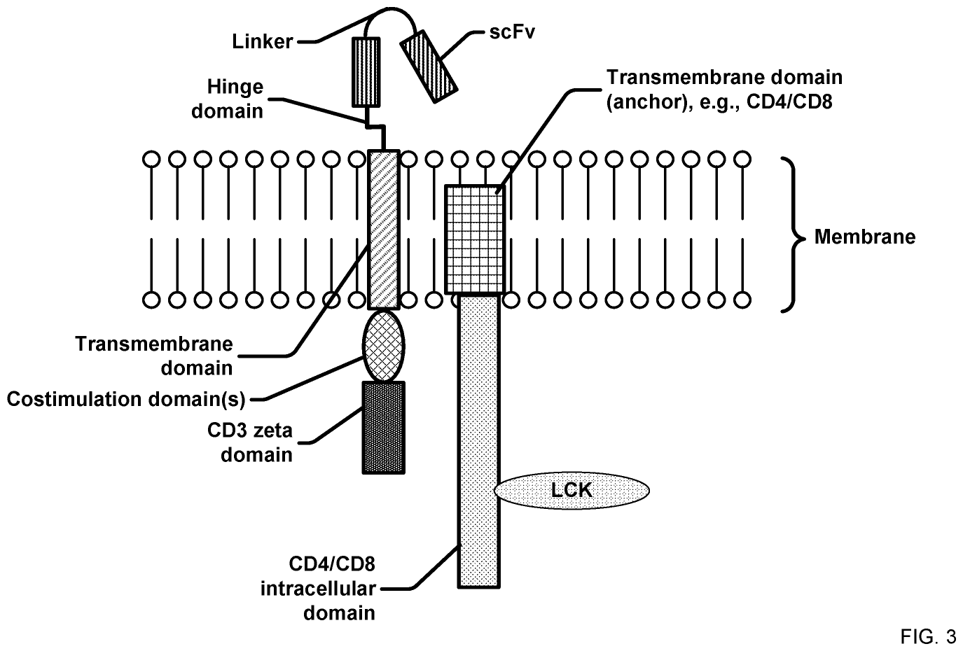

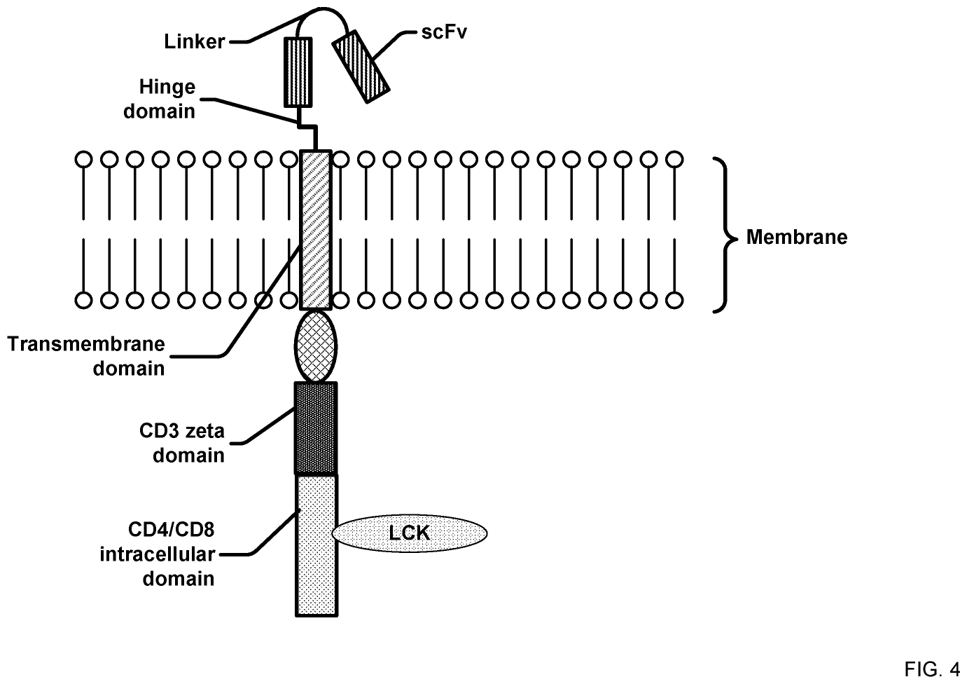

obtaining a polynucleotide encoding a CAR comprising a cytoplasmic domain of CD4 or CD8, for example, the CARs exemplified in FIGS. 2-9);
introducing the polynucleotide into a lymphocyte; and
contacting the lymphocyte with a cell or an antigen that the CAR targets or binds, thereby releasing IL-2, wherein an amount of IL-2 released is enhanced as compared to IL-2 release by the CAR without a CD4 or CD8 cytoplasmic domain.
60. The method of embodiment 59, wherein the cytoplasmic domain of CD4 comprises a LCK binding site.
61. The method of embodiment 58 or 59, wherein the lymphocyte is a NK or T cell.

Table 3 provides exemplary sequences. Related sequences are provided in this Application and Innovative Cellular Therapeutics' PCT Patent Applications Nos: PCT/CN2016/075061, PCT/CN2018/08891, and PCT/US19/13068, which are incorporated by reference in their entirety.

TABLE 3

| SEQ ID NO: | Notes |
|---|---|
| 1 | Modified CAR 1 |
| 2 | Modified CAR 2 |
| 3 | Modified CAR 3 |
| 4 | Modified CAR 4 |
| 5 | Modified CAR 5 |
| 6 | SP |
| 7 | CD8 hinge |
| 8 | CD8 transmembrane |
| 9 | CD8α cytoplasm |
| 10 | CD8β cytoplasm |
| 11 | residues of CD8α cytoplasm |
| 12 | CD4 hinge |
| 13 | CD4 transmembrane |
| 14 | CD4 cytoplasm part 1 incorporated into CAR |
| 15 | CD4 cytoplasm part 2 incorporated into CAR |
| 16 | MAGE-A4 CAR 41-BB |
| 17 | CD4 incorporated into CAR (1) |
| 18 | CD4 incorporated into CAR (2) |
| 19 | GCC scFv |
| 20 | CD4 incorporated into CAR (3) |
| 21 | CD8 incorporated into CAR (1) |
| 22 | CD8 incorporated into CAR (2) |
| 23 | CD8 incorporated into CAR (3) |
| 24 | Hinger and Transmembrane domains of CD8 incorporated into CAR |
| 25 | Modified CAR 6 6121 |
| 26 | Modified CAR 7 6122 |
| 27 | Modified CAR 8 6123 |
| 28 | CAR 9 8508 |
| 29 | AXL cytoplasm |
| 30 | CD2 cytoplasm |
| 31 | CD5 cytoplasm |
| 32 | CD44 cytoplasm |
| 33 | CD45 cytoplasm |
| 34 | CD122 cytoplasm |
| 35 | AXL cytoplasm incorporated into CAR |
| 36 | CD2 cytoplasm incorporated into CAR |
| 37 | CD4 cytoplasm incorporated into CAR |
| 38 | CD5 cytoplasm incorporated into CAR |
| 39 | CD8a cytoplasm incorporated into CAR |
| 40 | CD44 cytoplasm incorporated into CAR |
| 41 | CD45 cytoplasm incorporated into CAR |
| 42 | CD122 cytoplasm incorporated into CAR |

### Examples

Lentiviral vectors that encode individual CAR molecules were generated and transtected into T cells, which are described below. Techniques related to cell cultures, construction of cytotoxic T lymphocyte assay can be found in “Control of large, established tumor xenografts with genetically retargeted human T cells containing CD28 and CD137 domains,” PNAS, Mar. 3, 2009, vol. 106 no. 9, 3360-3365 and “Chimeric Receptors Containing CD137 Signal Transduction Domains Mediate Enhanced Survival of T Cells and Increased Antileukemic Efficacy In Vivo,” Molecular Therapy, August 2009, vol. 17 no. 8, 1453-1464, which are incorporated herein by reference in their entirety.

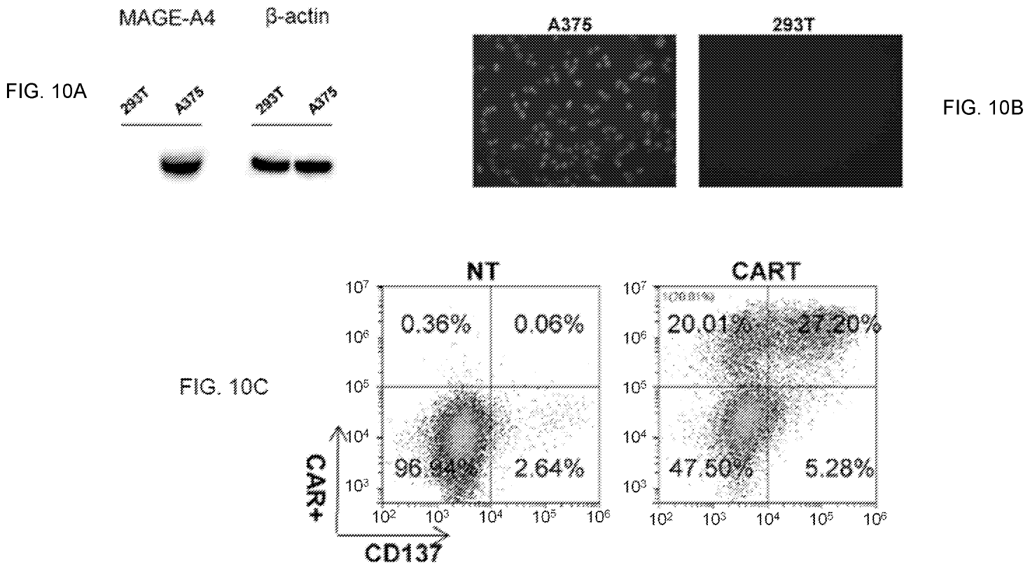

FIGS. 10-12 and 14-17 show various assays demonstrating that the incorporation of CD4 domain into CAR structure enhances CAR T cells' activation and cytokine release. FIGS. 10A-10C show the anti-tumor effect of CAR T cells.

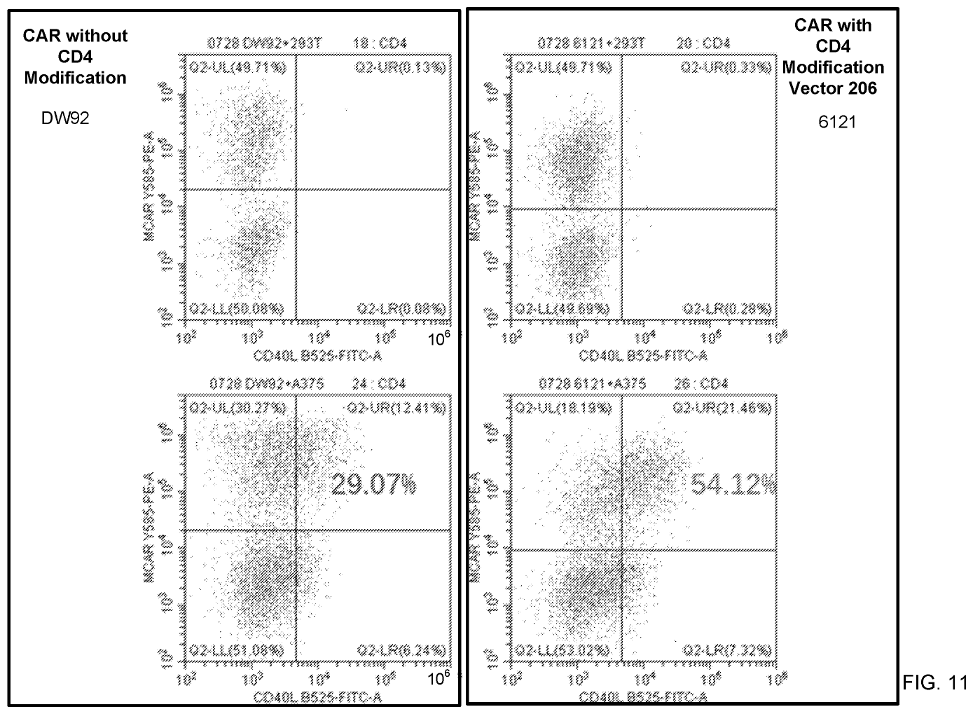

FIG. 10A shows the expression of CAR in human embryonic kidney (HEK) cell line 293T and human tumor cell line A375 with Western Blot. FIG. 10B shows the expression of CAR in cell line 293T and A375 with Immunofluorescence (IF) staining. FIG. 10C shows expression of CD137 in CAR-T cells co-cultured with the A375 cell lines. FIG. 11 shows flow cytometry results of the expression of CAR and CD40L on T cells that were co-cultured with substrates cells.

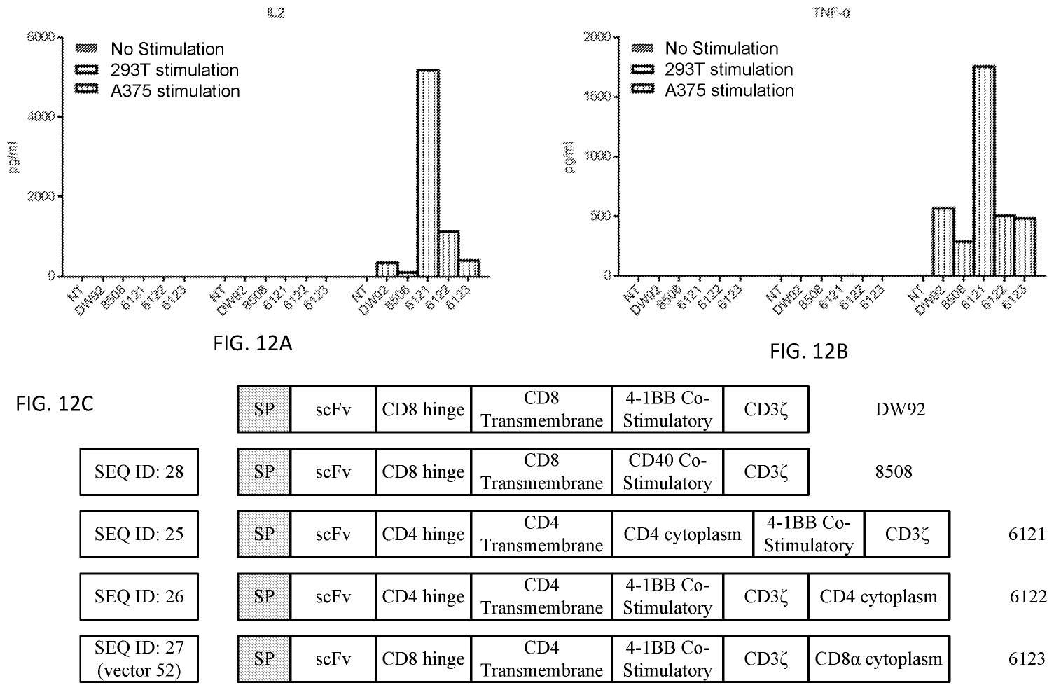

On day 0, peripheral blood was drawn from healthy volunteers and sorted to collect CD3⁺ T cells. Anti-CD3/CD28 dynabeads were added to the collected T cells at a 1:1 ratio. On day 1, the activated T cells were mixed with various vectors encoding various CARs binding MAGE-A4 peptide (see constructs in FIG. 12C). FIGS. 12A and 12B show cytokine release and cell expansion of CAR T cells co-cultured with substrate cells. As shown in FIGS. 10-12, the incorporation of CD4 into CAR structures enhance CAR T cells' activation and cytokine release as compared to conventional CAR T cells.

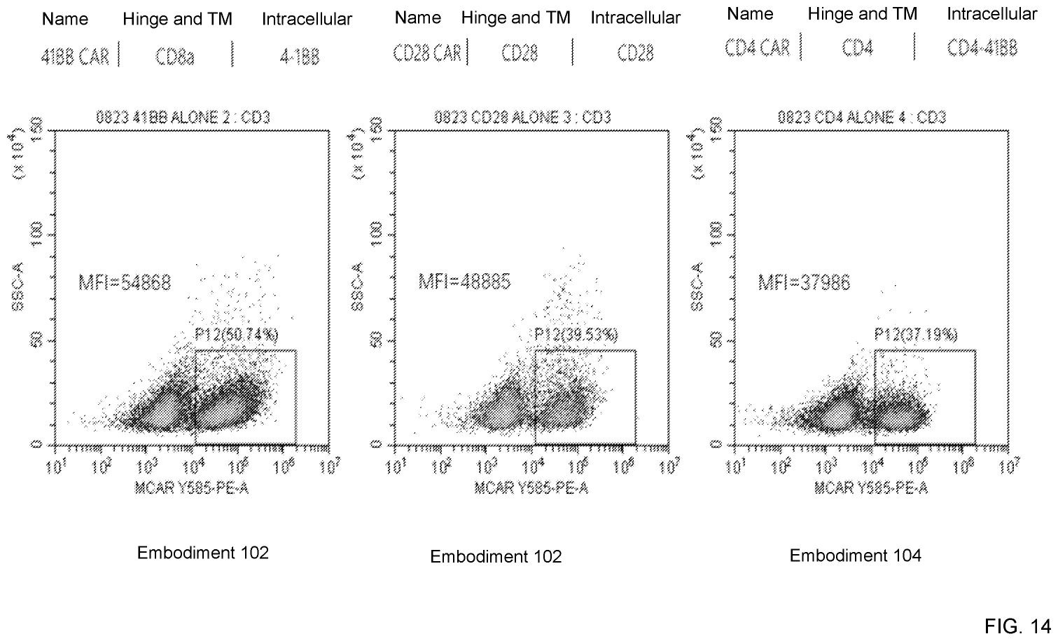

FIG. 14 shows flow cytometry results of various CARs being expressed on T cells.

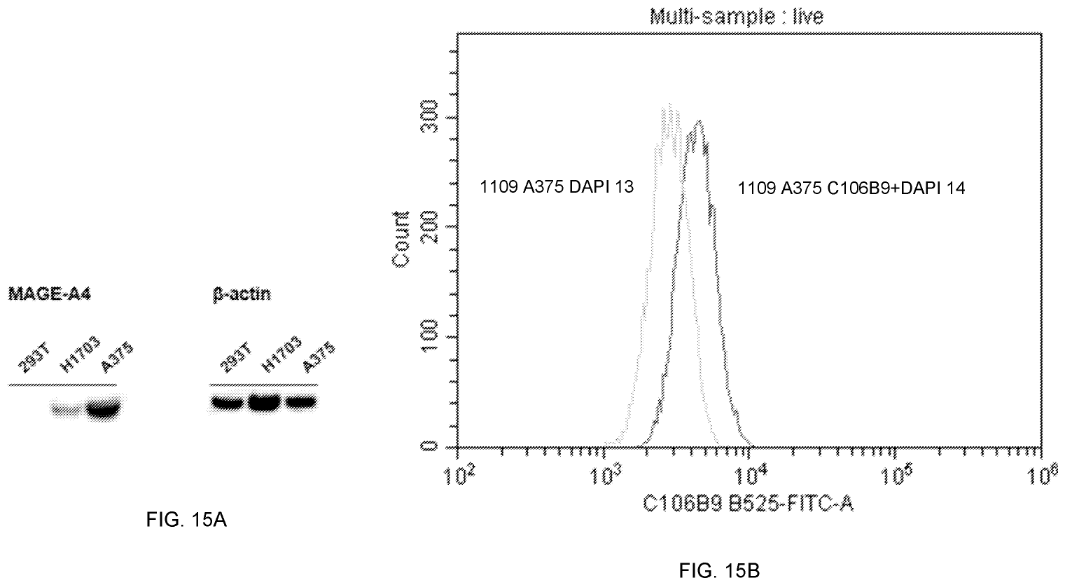

FIGS. 15A and 15B show expression of MAGE-A4 on A375 cells. West Blot (WB) was performed to detect the expression of MAGE-A4 and Anti-MHC-PEP tide complex. As shown in FIG. 15A, the antigen presentation level was low. A375 cells are natural melanoma cell lines, commonly used in PD 1/PD L1 or TIL experiments, and HLA A0201/MAGE-A4 positive.

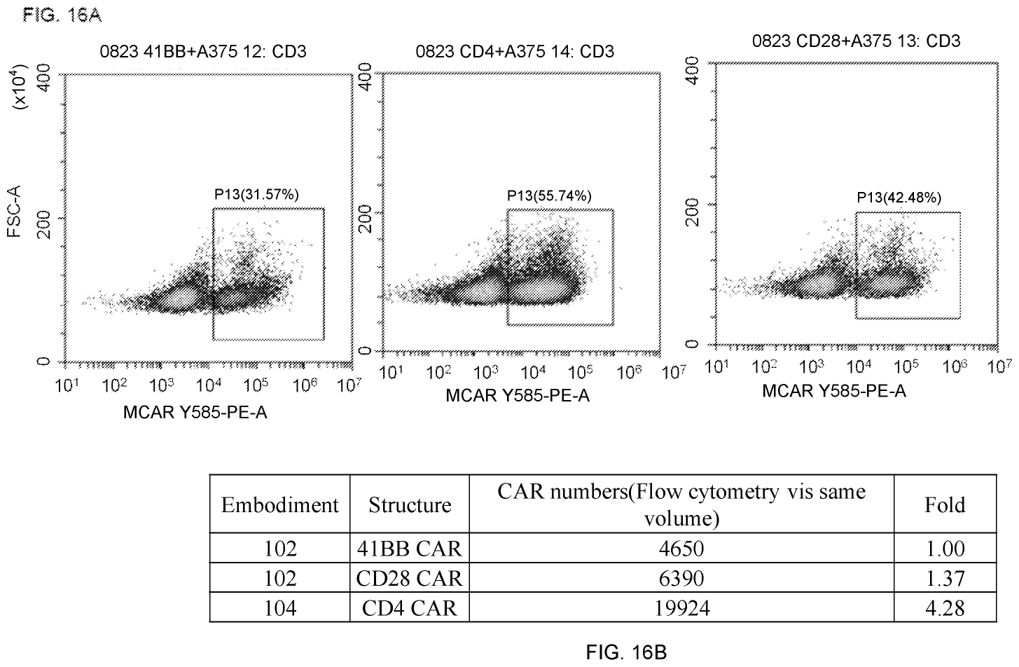

FIG. 16A shows flow cytometry results of the expansion of CAR T cells co-cultured with A375 cells for 96 hours. In combination with FIGS. 12A and 12B, FIG. 16A confirm that CAR T cells expressing CD4 incorporated CARs showed higher cell proliferation levels than CAR T cells expressing conventional second-generation CARs (i.e., Embodiment 102) after co-culturing with A375.

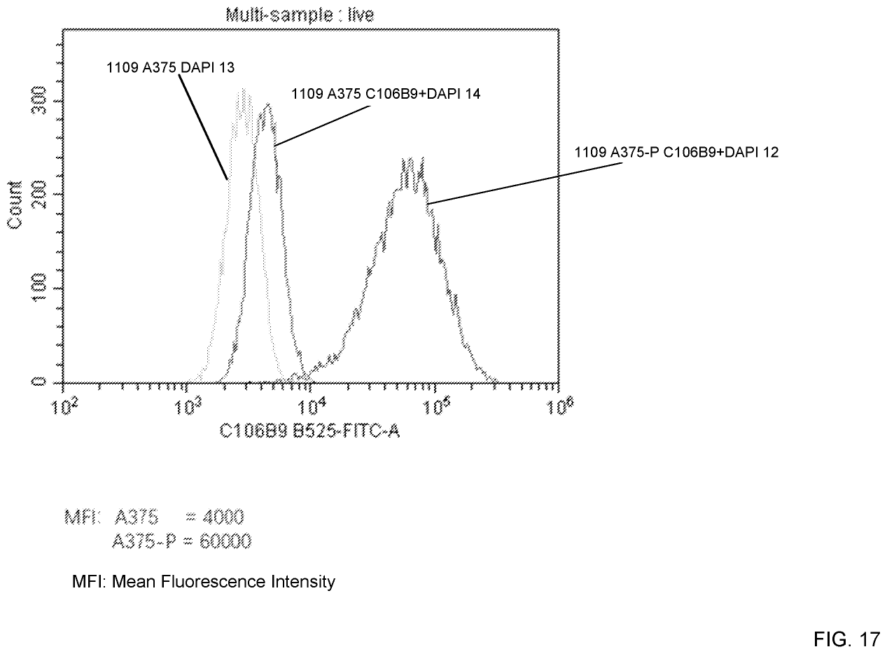

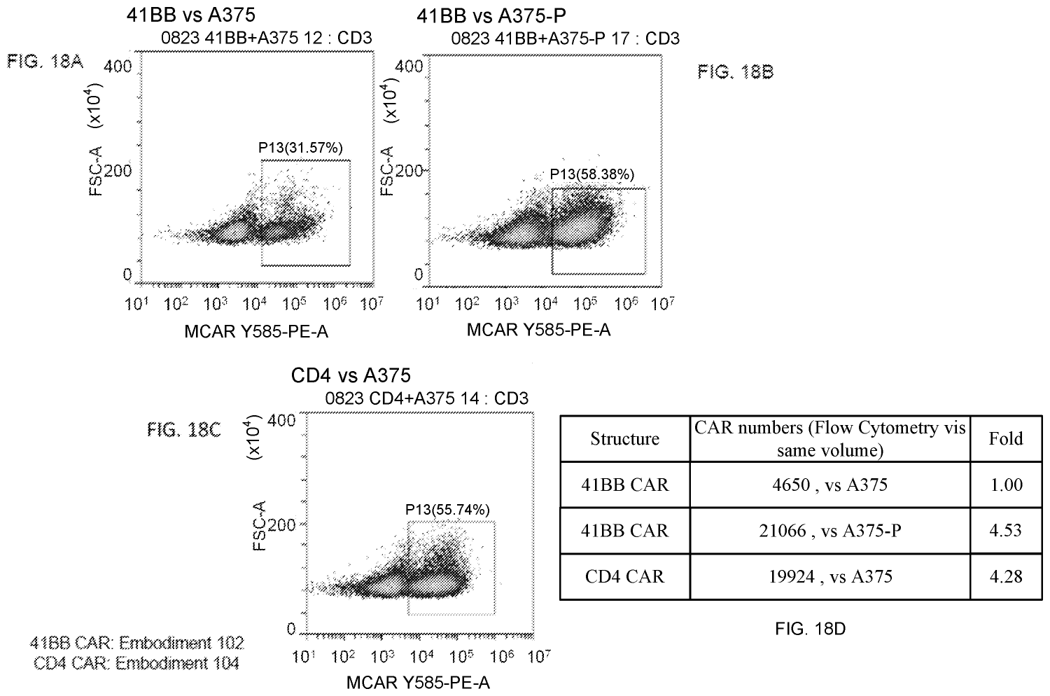

FIG. 17 shows flow cytometry results of expression of MAGE-A4 in A375 and A375-P cells. FIGS. 18A-18C show flow cytometry results of cell expansion of various CAR-T cells co-cultured with A375 cells or A375-P. Antigen concentration affects the level of CAR activation. As shown in FIGS. 17 and 18A-18C, CD4 CARs, for example, the dependence of CAR activation in Embodiment 104 on antigen concentrations and the presentation ability of the antigen of target cells appeared to be reduced. As described above, in the A375 model, T cells expressing conventional CARs, for example, Embodiment 102, show a lower level of response, a lower level of IL-2 released, and a lower rate of proliferation as compared to CAR T cells expressing modified CARs, for example, Embodiment 104. In the A375-P model, activation response and proliferation levels of T cells expressing conventional CARs are improved. These results indicate that LCK recruitment to CAR molecules may depend on the LCK binding region in the CAR structure and on the CAR molecule cluster formation.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1-13. (canceled)

14. A method of enhancing IL-2 release by T cells expressing a chimeric antigen receptor (CAR), the method comprising:
introducing the polynucleotide into a T cell to obtain a CAR T cell, the polynucleotide encoding the CAR comprising an extracellular domain, a transmembrane domain, and an intracellular domain comprising a cytoplasmic domain of CD4 or CD8 and a stimulatory signaling region; and
contacting the CAR T cell with a tumor cell or tumor antigen that the CAR T cell binds, thereby releasing IL-2, wherein an amount of IL-2 released is enhanced as compared to IL-2 released by a CAR T cell without a cytoplasmic domain of CD4 or CD8.

15. The method of claim 14, wherein the CAR comprises SEQ ID NO: 18 and SEQ ID NO: 19, or the CAR comprises SEQ ID NO: 3.

16. The method of claim 14, wherein the cytoplasmic domain comprises SEQ ID NO: 9, 11, 14, or 15.

17. The method of claim 14, wherein the CAR T cell comprises a polynucleotide encoding a therapeutic agent comprising IL-1P, IL-2, IL-4, IL-5, IL-6, IL-8, IL-10, IL-12, IL-13, IL-15, IL-17, IL-1Ra, IL-2R, IFN-γ, IFN-γ, MIP-In, MIP-IP, MCP-1, TNFα, GM-CSF, CCL19, or MIP-Iα, GCSF, CXCL9, CXCL10, CXCR factors, VEGF, RANTES, EOTAXIN, EGF, HGF, FGF-P, CD40, CD40L, ferritin, or any combination thereof.

18. The method of claim 17, wherein the polynucleotide encoding the therapeutic agent further encodes a VHL-interaction domain of HIF1α.

19. The method of claim 14, wherein the CAR T cell comprises a polynucleotide encoding a dominant negative form of an inhibitory immune checkpoint molecule or a receptor thereof.

20. The method of claim 14, wherein the CAR T cell has a reduced expression of endogenous TRAC gene.

21. The method of claim 14, wherein the cytoplasmic domain comprises SEQ ID NO: 9 or 14.

22. The method of claim 14, wherein the cytoplasmic domain comprises SEQ ID NO: 11 or 15.

23. The method of claim 14, wherein the intracellular domain further comprises a co-stimulatory signaling region, and the cytoplasmic domain of CD4 is between the transmembrane domain of the CAR and the co-stimulatory signaling region.

24. The method of claim 14, wherein the intracellular domain further comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

25. The method of claim 14, wherein the CAR binds GCC, TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, Lewis Y, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, ETV6-AML, sperm protein 17, XAGE1, Tie 2, MAD-CT-1, MAD-CT-2, Fos-related antigen 1, p53, p53 mutant, prostein, survivin, telomerase, PCTA-1 (Galectin 8), MelanA (MART1), Ras mutant, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase (hTERT), RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, or IGLL1.

26. The method of claim 14, wherein the intracellular domain further comprises CD3 zeta domain.

27. The method of claim 14, wherein the cytoplasmic domain of CD4 comprises a LCK binding.

28. The method of claim 14, wherein the intracellular domain of the CAR comprises SEQ ID NO: 17 or 20.

29. The method of claim 14, wherein the CAR comprises SEQ ID NO: 3 or 18 and a scFv targeting a solid tumor antigen.
